SEQUENCE LISTING (2 sequences)

SEQ ID NO: 1 - protein, 249 aa, synthetic construct
APSFDCGKPQVEPKKCPGRVVGGCVAHPHSWPWQVSLRTRFGMHFCGGTLISPEWVLTAAHCLEKSPRPSSYKVILGAHQEVNLEPHVQEIEVSRLFLEPTRKDIALLKLSSPAVITDKVIPACLPSPNYVVADRTECFITGWGETQGTFGAGLLKEAQLPVIENKVCNRYEFLNGRVQSTELCAGHLAGGTDSCQGDSGGPLVCFEKDKYILQGVTSWGLGCARPNKPGVYVRVSRFVTWIEGVMRNN

SEQ ID NO: 2 - protein, 810 aa, Homo sapiens
MEHKEVVLLLLLFLKSGQGEPLDDYVNTQGASLFSVTKKQLGAGSIEECAAKCEEDEEFTCRAFQYHSKEQQCVIMAENRKSSIIIRMRDVVLFEKKVYLSECKTGNGKNYRGTMSKTKNGITCQKWSSTSPHRPRFSPATHPSEGLEENYCRNPDNDPQGPWCYTTDPEKRYDYCDILECEEECMHCSGENYDGKISKTMSGLECQAWDSQSPHAHGYIPSKFPNKNLKKNYCRNPDRELRPWCFTTDPNKRWELCDIPRCTTPPPSSGPTYQCLKGTGENYRGNVAVTVSGHTCQHWSAQTPHTHNRTPENFPCKNLDENYCRNPDGKRAPWCHTTNSQVRWEYCKIPSCDSSPVSTEQLAPTAPPELTPVVQDCYHGDGQSYRGTSSTTTTGKKCQSWSSMTPHRHQKTPENYPNAGLTMNYCRNPDADKGPWCFTTDPSVRWEYCNLKKCSGTEASVVAPPPVVLLPDVETPSEEDCMFGNGKGYRGKRATTVTGTPCQDWAAQEPHRHSIFTPETNPRAGLEKNYCRNPDGDVGGPWCYTTNPRKLYDYCDVPQCAAPSFDCGKPQVEPKKCPGRVVGGCVAHPHSWPWQVSLRTRFGMHFCGGTLISPEWVLTAAHCLEKSPRPSSYKVILGAHQEVNLEPHVQEIEVSRLFLEPTRKDIALLKLSSPAVITDKVIPACLPSPNYVVADRTECFITGWGETQGTFGAGLLKEAQLPVIENKVCNRYEFLNGRVQSTELCAGHLAGGTDSCQGDSGGPLVCFEKDKYILQGVTSWGLGCARPNKPGVYVRVSRFVTWIEGVMRNN

(19) United States
(12) Patent Application Publication
(10) Pub. No.: US 2026/0306549 A1
(43) Pub. Date: Oct. 8, 2026

(54) PLASMIN-INDUCED INTERNAL LIMITING MEMBRANE DIGESTION FACILITATES DONOR RETINAL GANGLION CELL TRANSPLANTATION

(71) Applicant: University of Pittsburgh - Of the Commonwealth System of Higher Education, Pittsburgh, PA (US)

(72) Inventors: Kun-Che Chang, Pittsburgh, PA (US); Dimitrios Stavropoulos, Pittsburgh, PA (US)

(73) Assignee: University of Pittsburgh - Of the Commonwealth System of Higher Education, Pittsburgh, PA (US)

(21) Appl. No.: 19/640,277

(22) Filed: Apr. 6, 2026

Related U.S. Application Data
(60) Provisional application No. 63/785,503, filed on Apr. 8, 2025.

(51) Int. Cl. A61K 35/30; A61K 9/00; A61K 31/4178; A61K 31/498; A61K 31/5377; A61K 31/5575; A61K 38/48; A61K 45/06; A61P 27/02

(52) U.S. Cl. CPC A61K 35/30; A61K 9/0048; A61K 31/4178; A61K 31/498; A61K 31/5377; A61K 31/5575; A61K 38/484; A61K 45/06; A61P 27/02; C12Y 304/21007

(57) ABSTRACT

Methods are disclosed for increasing integration of retinal ganglion cells (RGCs) in a retina of a subject. Methods are also disclosed for treating glaucoma or optic neuropathy in a subject. These methods include administering an effective amount of ocriplasmin, plasmin, or plasminogen to an eye of the subject; and transplanting an effective amount of RGCs into the eye of the subject, thereby increasing the integration of RGCs in the retina in the eye of the subject and/or treating the glaucoma or optic neuropathy in the eye of a subject.

### CROSS REFERENCE TO RELATED APPLICATIONS

This claims the benefit of U.S. Provisional Application No. 63/785,503, filed Apr. 8, 2025, which is incorporated herein by reference in its entirety.

### FIELD

This relates to the field of transplantation, specifically to the use of plasmin to digest the internal limiting membrane (ILM) in the retina, and its use to increase the integration of transplanted retinal ganglion cells (RGCs).

### REFERENCE TO AN ELECTRONIC SEQUENCE LISTING

The contents of the electronic sequence listing, file named Sequence.xml, 3,722 bytes, dated Jan. 28, 2026, which is submitted as a XML file, is herein incorporated by reference in its entirety.

### BACKGROUND

RGCs are the first neurons in the visual pathway, solely responsible for transmitting visual signals from the retina to the brain via the optic nerve. Damage or degeneration of RGCs can result in vision impairment and loss, as observed in conditions like glaucoma and optic neuropathies. Various strategies are being explored to restore RGC function, including cell replacement therapy and gene therapy. Gene therapy involves delivering exogenous genetic material into cells using viral or non-viral vectors to modify or restore the function of a specific gene, with the goal of enhancing the survival and regeneration of RGCs. In cases where a significant portion of the RGCs is lost, cell replacement therapy also emerges as a potential treatment avenue. Given the limited availability of human RGCs, inducing the differentiation of human stem cells into RGCs offers a means of generating replacement cells for transplantation into the impaired retina. While several studies have demonstrated the successful transplantation of human stem cell-derived RGCs into various animal models, the challenge of low donor cell survival post-transplantation persists. Consequently, devising methods to enrich transplantable RGCs and enhance donor cell survival and integration within the host eye remains a requirement for advancing cell replacement therapy. Disclosed herein are methods that meet this need.

### SUMMARY

Methods are disclosed for increasing integration of RGCs in a retina of a subject. Methods are also disclosed for treating glaucoma or optic neuropathy in a subject. These methods include administering an effective amount of ocriplasmin, plasmin, or plasminogen to an eye of the subject; and transplanting an effective amount of RGCs into the eye of the subject, thereby increasing the integration of RGCs in the retina in the eye of the subject and/or treating the glaucoma or optic neuropathy in the eye of a subject.

The foregoing and other objects, features, and advantages of the invention will become more apparent from the following detailed description, which proceeds with reference to the accompanying figures.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1B. Intravitreal administration of the optimal plasmin dose does not cause immune cell infiltration or microglia activation. (FIG. 1A) No immune cell infiltration was observed in the vitreous cavity of the plasmin-treated eyes. A representative image at the bottom of FIG. 1A serves as a reference, illustrating lipopolysaccharide-induced immune cells infiltration. (FIG. 1B) Intravitreal injection of 4.6 μg of plasmin resulted in a significant increase in the number of microglia cells in the retina, especially in the ganglion cell layer, in contrast to the 2.3 μg dose which induced no microglia reactivity. Mean±standard error of the mean (SEM) shown; ns=not significant; ****p<0.0001, ***p<0.001, **p<0.01 by one-way ANOVA and post hoc t-test with Tukey correction. N=4 per group (A) N≥9 per group (B).

FIGS. 2A-2C. Plasmin treatment is capable of disrupting the internal limiting membrane without damaging RGCs. (FIG. 2A) Representative pictures showing ILM immunostained using laminin, disrupted by both 2.3 μg and 4.6 μg of plasmin treatment. A representative image at the top of FIG. 2A illustrates a non-injected negative control. (FIG. 2B) Retinal sections demonstrating that the number of Brn3a+ RGCs was not reduced by either the 2.3 μg or the 4.6 μg plasmin treatment. (FIG. 2C) Retinal flatmounts showing that the Brn3a+ RGC population was maintained across all three groups. Mean+SEM shown; ns=not significant; ****p<0.0001 by one-way ANOVA and post hoc t-test with Tukey correction (A and B). N>4 per group.

FIGS. 3A-3F. RGC function is reduced in the 4.6 μg plasmin treatment group, while ganglion cell complex thickness demonstrated no changes (FIG. 3A) Graph illustrating the generated pERG responses from the plasmin-injected and PBS-injected eyes 2 weeks after treatment. The use of 4.6 μg of plasmin treatment caused reduction of RGCs and cone function while with the 2.3 μg dose RGCs maintained their functional integrity. (FIG. 3B) pERG responses 2 weeks after 6-aminohexanoic acid injections showing no significant difference in total amplitude indicating that the observed effects are not driven by 6-aminohexanoic acid itself. (FIG. 3C) One-month post-treatment, pERG revealed a consistent reduction in RGC function in the 4.6 μg group. (FIG. 3D) Scotopic a-wave analysis revealed a significant reduction in the 4.6 μg group compared to both other groups function, while scotopic b-wave analysis showed no statistically significant differences. (FIG. 3E) OCT images collected two weeks post-treatment showing retinal structure (top) and the corresponding RGC axon bundles (bottom). GCC and whole retina thickness demonstrated no significant changes in the plasmin treatment groups compared to PBS. (FIG. 3F) Oscillatory potentials showed no significant differences between groups, with a non-significant trend toward reduction in the 4.6 μg group. Mean±SEM shown; ns=not significant; *p<0.05 by one-way ANOVA and post hoc t-test with Tukey correction. N≥5 per group.

FIGS. 4A-4E. The survival of transplanted cells is increased one week after transplantation in the plasmin-treated eyes. (FIG. 4A) Day 60 human retinal organoid expressing tdTomato fluorescence in the center, indicating Brn3b expression and RGC presence. (FIG. 4B) More tdTomato+ cells were observed on plasmin-treated retinas one week after transplantation indicating increased donor cell survivability. (FIG. 4C) No difference in donor cell survivability was observed between the two groups one month after transplantation. (FIG. 4D) Z-projections and 3D representations of a plasmin-treated retina, 1 week after transplantation, showing tdTomato+donor cells extending their axons in the same layer where other non-donor DAPI+ cells are. (FIG. 4E) Representative images of a plasmin-treated retina, 1 month after transplantation, showing colocalization of tdTomato fluorescence and RBPMS staining in donor cells confirming their RGC identity. Mean±SEM shown; **p<0.01 by unpaired student's t-test. N≥4 per group.

### DETAILED DESCRIPTION

It is disclosed herein that the efficacy of plasmin, a vitreolytic enzyme analogous to Ocriplasmin, which is a truncated recombinant form of plasmin and a previously FDA-approved drug (Sheridan et al., Nat Biotechnol, 30, 1158. 2012), was evaluated. Plasmin is a protease capable of liquefying the vitreous and can also break down laminin and fibronectin, two key proteins that help the posterior hyaloid membrane adhere to the ILM. This breakdown assists in inducing posterior vitreous detachment by weakening the links between these components of the vitreoretinal interface and the ILM (Nazari et al., Pharmacologic vitreolysis. J Ophthalmic Vis Res, 5, 44-52, 2010). An efficient plasmin treatment dose was identified that effectively disrupts the ILM without inducing immune cell infiltration, microglial reactivity or damage to RGCs. The results provide an improved method to enhance donor RGCs survival after transplantation by ILM disruption, without causing ocular inflammation.

Ocriplasmin, marketed under the brand name Jetrea, is a recombinant protease that targets fibronectin and laminin, which are integral components of the vitreoretinal interface. Approved by the FDA in 2012 (Sheridan et al., Nat Biotechnol, 30, 1158. 2012), it was utilized to address symptomatic vitreomacular adhesion by breaking down the proteins responsible for tethering the vitreous to the macula (Shaikh et al., Semin Ophthalmol, 32, 52-55, 2017). This action facilitates the detachment of the vitreous from the retina (Neffendorf et al., Cochrane Database Syst Rev, 10, Cd011874, 2017). While the use of Ocriplasmin may lead to adverse effects such as vitreous floaters and photopsia, the majority are mild to moderately severe, and transient in nature (Neffendorf et al., Cochrane Database Syst Rev, 10, Cd011874, 2017; Dugel et al., Ophthalmology, 123, 2232-47, 2016). Although Ocriplasmin was discontinued in 2020, plasmin—a non-specific serine protease with fibrinolytic properties—remains a key enzyme with similar biological activity. In addition to its role in fibrinolysis, plasmin can hydrolyze various glycoproteins, including laminin and fibronectin, which are essential components of the vitreoretinal interface. Plasmin also exhibits the capacity to liquefy the vitreous humor (Nazari et al., Ophthalmic Vis Res, 5, 44-52, 2010), potentially fostering improved attachment of donor cells to the host retina.

As the basement membrane of Müller cells in the retina, the ILM represents a structural interface between the retina and the vitreous (Wiedemann et al., Int J Ophthalmol, 16, 837-840, 2023). While integral for the early development of the optic nerve and retina, the ILM may undergo pathological changes such as increased thickness or proliferation of epiretinal cells (Wiedemann et al., Int J Ophthalmol, 16, 837-840, 2023). These changes may subsequently exert tractional forces on the retina, particularly as individuals age or in the presence of pathological conditions such as retinal ischemia or inflammation (Razaei et al., J Ophthalmic Vis Res, 9, 215-22, 2014; Fung et al., Clin Exp Ophthalmol, 49, 289-308, 2021).

Removal of ILM is considered an important procedure for enhancing RGC survival and integration after transplantation (Johnson et al., Ophthalmol Sci, 3, 100390, 2023; Soucy et al., Mol Neurodegener, 18, 64, 2023). A recent ex vivo study on retinal explants reported that mechanical depletion of the ILM in the mouse eye promotes the donor cells targeting the inner retina (Do et al., Invest Ophthalmol Vis Sci, 64, 25, 2023). Surgical peeling of the ILM is a common procedure beneficial for repairing traction maculopathies affecting the vitreoretinal interface, such as macular pucker (Fung et al., Clin Exp Ophthalmol, 49, 289-308, 2021), retinal detachment (Fung et al., Cochrane Database Syst Rev, 3, CD015514, 2024) and macular holes (Arda et al., Surv Ophthalmol., 2024). However, vitrectomy combined with ILM peeling may lead to various complications, such as changes in intraocular pressure, progression of cataracts or dislocation of lenses, iatrogenic retinal tears or detachment, alterations in retinal pigment epithelium, visual field impairments, vitreous hemorrhage, and endophthalmitis (Asencio-Duran et al., J Ophthalmol, 2015, 467814, 2015; Gupta et al., Ophthalmic Surg Lasers Imaging, 38, 270-5, 2007; Park et al., Ophthalmology, 102, 775-81, 1995). In response to these challenges, the process of enzymatic digestion, which hydrolyzes and softens the ILM, presents itself as an alternative method for ILM removal, offering a solution that mitigates the risk of inducing complications commonly associated with surgical removal techniques. This enzymatic approach holds promise in providing a safer means of addressing ILM-related concerns.

### Terms

Unless otherwise noted, technical terms are used according to conventional usage. Definitions of many common terms in molecular biology may be found in Krebs et al. (eds.), Lewin's genes XII, published by Jones & Bartlett Learning, 2017. As used herein, the singular forms “a,” “an,” and “the,” refer to both the singular as well as plural, unless the context indicates otherwise. For example, the term “a protein” includes single or plural proteins and can be considered equivalent to the phrase “at least one protein.” As used herein, the term “comprises” means “includes.” Unless otherwise indicated “about” indicates within five percent. It is further to be understood that any and all base sizes or amino acid sizes, and all molecular weight or molecular mass values, given for nucleic acids or polypeptides are approximate, and are provided for descriptive purposes, unless otherwise indicated. Although many methods and materials similar or equivalent to those described herein can be used, particular suitable methods and materials are described below. In case of conflict, the present specification, including explanations of terms, will control. In addition, the materials, methods, and examples are illustrative only and not intended to be limiting. To facilitate review of the various aspects, the following explanations of terms are provided:

Animal: Living multi-cellular vertebrate organisms, a category that includes, for example, mammals and birds. The term mammal includes both human and non-human mammals. Similarly, the term “subject” includes both human and veterinary subjects.

Cell: A structural and functional unit of an organism that can replicate independently, is enclosed by a membrane, and contains biomolecules and genetic material. Cells used herein may be naturally-occurring cells or artificially modified cells (e.g., fusion cells, genetically modified cells, etc.).

The term “cell population” refers to a group of cells, typically of a common type. The cell population can be derived from a common progenitor or may comprise more than one cell type. An “enriched” cell population refers to a cell population derived from a starting cell population (e.g., an unfractionated, heterogeneous cell population) that contains a greater percentage of a specific cell type, such as macular, central or peripheral RPE cells, than the percentage of that cell type in the starting population. The cell populations may be enriched for one or more cell types and depleted of one or more cell types.

Glaucoma: An eye disorder characterized by retinal ganglion cell death, excavation of the optic nerve head and gradual loss of the visual field. An abnormally high intraocular pressure is commonly known to be detrimental to the eye and is one of the main risk factors in glaucoma. In glaucoma patients, high intraocular pressure can result in degenerative changes in the retina. “Ocular hypertension” refers to clinical situation in individuals with an abnormally high intraocular pressure without any manifestation of defects in the visual field or optic nerve head. Individuals with ocular hypertension carry the risk of conversion to glaucoma with the risk being correlated to higher intraocular pressure measurements.

Glaucoma can be divided into open-angle form and the closed-angle forms and further classified into acute and chronic forms. There also is a normal-tension glaucoma. The glaucoma can be a primary or a secondary glaucoma. More than 80% of all glaucoma cases are chronic open angle glaucoma (COAG), also called primary open angle glaucoma. Any of these forms of glaucoma can be treated using the methods disclosed herein.

“Primary angle closure glaucoma” is caused by contact between the iris, trabecular meshwork, and peripheral cornea which in turn obstructs outflow of the aqueous humor from the eye. This contact between iris and trabecular meshwork (TM) may gradually damage the function of the meshwork until it fails to keep pace with aqueous production, and the pressure rises. In over half of all cases, prolonged contact between iris and TM causes the formation of synechiae (effectively “scars”). These cause permanent obstruction of aqueous outflow. In some cases, pressure may rapidly build up in the eye, causing pain and redness (symptomatic, or so-called “acute” angle closure). In this situation, the vision may become blurred, and halos may be seen around bright lights. Accompanying symptoms may include a headache and vomiting. Diagnosis can made from physical signs and symptoms: pupils mid-dilated and unresponsive to light, cornea edematous (cloudy), reduced vision, redness, and pain. However, the majority of cases are asymptomatic. Prior to the very severe loss of vision, these cases can only be identified by examination, generally by an eye care professional.

“Primary open-angle glaucoma” occurs when optic nerve damage results in a progressive loss of the visual field. Not all people with primary open-angle glaucoma have eye pressure that is elevated beyond normal. The increased pressure is caused by the blockage of the aqueous humor outflow pathway. Because the microscopic passageways are blocked, the pressure builds up in the eye and causes imperceptible very gradual vision loss. Peripheral vision is affected first, but eventually the entire vision will be lost if not treated. Diagnosis can be made by looking for cupping of the optic nerve and measuring visual field. Prostaglandin agonists work by opening uveoscleral passageways.

Other forms of glaucoma are developmental glaucoma and secondary glaucoma, which can occur after uveitis, iridocyclitis, intraocular hemorrhage, trauma, or an intraocular tumor. Any form of glaucoma can be treated using the methods disclosed herein.

The death of RGCs occurs in glaucoma. Methods are disclosed herein for increasing the survival of RGCs.

Immunosuppressant agent: Any type of drug or treatment regimen that weakens a subject's immune system, such as to reduce the risk of transplant rejection, to prevent/treat graft versus host disease (GVHD), or to treat an autoimmune disease. Immunosuppressant agents include, but are not limited to, corticosteroids (e.g., prednisone), calcineurin inhibitors (e.g., tacrolimus, cyclosporine), inosine monophosphate dehydrogenase (IMDH) inhibitors (e.g., mycophenolate mofetil), Janus kinase inhibitors (e.g., XELIJANZ®), mechanistic target of rapamycin (mTOR) inhibitors (e.g., sirolimus), anti-metabolites (e.g., mycophenolate mofetil (MMF) or mycophenolic acid (MPA)), and biologics (e.g., basiliximab, adalimumab, infliximab, alemtuzumab, belatacept).

Integration (of cells): A process allowing cells to adhere, communicate, and cooperate within tissues. Integration involves cell adhesion and communication of transplanted cells with other endogenous cells within the tissue. Integration of retinal cells is the incorporation of transplanted RGCs into the ganglion cell layer of the retina, optionally including neurite extension and synapse formation.

Internal Limiting Membrane (ILM): A thin and transparent acellular membrane on the surface of the retina acting as a boundary between the retina and the vitreous body. It is composed of networks of secretory extracellular matrix proteins such as collagen IV and laminins, while it is formed by astrocytes and the end feet of Müller cells. It is separated from the vitreous body by a basal lamina.

Intraocular Administration: Administering agents directly into the eye, for example by delivery into the vitreous or anterior chamber. Indirect intraocular delivery (for example by diffusion through the cornea) is not direct administration into the eye.

Intraocular Pressure: The pressure of the fluid that fills the eye globe, the aqueous humor, which is determined by the interplay between the rate of aqueous humor production inside the eye and the resistance to aqueous outflow as it exits the eye through the anterior chamber angle towards Schlemm's canal. In the human eye, the rate of aqueous formation is 2.54 μ/minute while that in the rabbit eye is approximately 3 to 4 μL/minute. Normal IOP measurements in the human eye, according to widely acceptable consensus, range between 10 and 20 mm of mercury, with an average of 15.5 mm.

Intravitreal administration: Administering agents into the vitreous cavity. The vitreous cavity is the space that occupies most of the volume of the core of the eye with the lens and its suspension system (the zonules) as its anterior border and the retina and its coating as the peripheral border. Intravitreal administration can be accomplished by injection, pumping, or by implants.

Ocriplasmin: A protein of 249 amino acid residues that consists of 2 peptide chains. The first peptide is 19 amino acid residues long. The second is 230 amino acid residues long. The peptides are linked together by 2 disulfide bonds between C6: C124 and C16: C24 residues according to the residue numbering presented below. Four other disulfide bonds link the cysteine residues in the second peptide as C46: C62, C138: C205, C168: C184 and C195: C223. Ocriplasmin does not contain O- or N-linked glycosylation. The ocriplasmin amino acid sequence is presented below:

(SEQ ID NO: 1)
APSFDCGKPQVEPKKCPGRVVGGCVAHPHSWPWQVSLRTRFGMHFCGGT
LISPEWVLTAAHCLEKSPRPSSYKVILGAHQEVNLEPHVQEIEVSRLFL
EPTRKDIALLKLSSPAVITDKVIPACLPSPNYVVADRTECFITGWGETQ
GTFGAGLLKEAQLPVIENKVCNRYEFLNGRVQSTELCAGHLAGGTDSCQ
GDSGGPLVCFEKDKYILQGVTSWGLGCARPNKPGVYVRVSRFVTWIEGV
MRNN

Recombinant human ocriplasmin is produced in the transformed yeast Pichia pastoris by standard fermentation technology and purified using standard biotechnology purification procedures. Fermentation of P. pastoris results in the secretion of microplasminogen which is purified and converted to ocriplasmin using an activation chromatography step; the active ocriplasmin can be further purified and formulated. A product monograph is available on the internet at pdf.hres.ca/dpd_pm/00048523.PDF, dated Sep. 27, 2018, incorporated herein by reference. It is available as a solution, 0.125 mg/0.1 mL (0.375 mg/0.3 mL).

Optic Neuropathy: Damage to the optic nerve. The main symptom is loss of vision, with colors appearing subtly washed out in the affected eye. A pale disc is characteristic of long-standing optic neuropathy. In ischemic optic neuropathies, there is insufficient blood flow (ischemia) to the optic nerve. Anterior ischemic optic neuropathy (AION) includes diseases that affect the optic nerve head and cause swelling of the optic disc. These diseases often cause sudden rapid visual loss in one eye. Posterior ischemic optic neuropathy is a syndrome of sudden visual loss with optic neuropathy without initial disc swelling with subsequent development of optic atrophy. Radiation optic neuropathy (RON) is also thought to be due to ischemia of the optic nerve that occurs 3 months to 8 or more years after radiation therapy to the brain and orbit. Optic neuropathy can also be caused by a compression injury or trauma. Heavy metal toxicity, methanol poisoning and ethylene glycol poisoning also can cause optic neuropathy. Optic neuritis is inflammation of the optic nerve, which is associated with swelling and destruction of the myelin sheath covering the optic nerve.

Pharmaceutically acceptable carriers: The pharmaceutically acceptable carriers useful in this invention are conventional. Remington's Pharmaceutical Sciences, by E. W. Martin, Mack Publishing Co., Easton, PA, 15th Edition (1975), describes compositions and formulations suitable for pharmaceutical delivery of the fusion proteins herein disclosed.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Pharmaceutical agent: A chemical compound or composition capable of inducing a desired therapeutic or prophylactic effect when properly administered to a subject or a cell. “Incubating” includes a sufficient amount of time for a drug to interact with a cell. “Contacting” includes incubating an agent, such as plasmin, plasminogen or ocriplasmin, with a cell.

Plasmin: A serine protease produced from the zymogen plasminogen. Plasminogen is released from the liver into the systemic circulation. Two major glycoforms of plasminogen are present in humans-type I plasminogen contains two glycosylation moieties (N-linked to N289 and O-linked to T346), whereas type II plasminogen contains only a single O-linked sugar (O-linked to T346). Plasminogen adopts a closed, activation-resistant conformation. Full length plasminogen comprises seven domains. In addition to a C-terminal chymotrypsin-like serine protease domain, plasminogen contains an N-terminal Pan Apple domain (PAp) together with five Kringle domains (KR1-5). The Pan-Apple domain contains important determinants for maintaining plasminogen in the closed form, and the kringle domains are responsible for binding to lysine residues present in receptors and substrates. Upon binding to clots, or to the cell surface, plasminogen adopts an open form that can be converted into active plasmin by a variety of enzymes, including tissue plasminogen activator (tPA), urokinase plasminogen activator (uPA), kallikrein, and factor XII (Hageman factor). The nucleic acid sequence of an mRNA encoding human plasminogen, are available at GENBANK® Accession No. NM_001168338.1, Oct. 6, 2024; GENBANK® Accession No. NM_000301.5, Oct. 12, 2024; the amino acid sequence of human plasminogen is available at GENBANK® Accession No. NP_000292.1, Oct. 12, 2024 and GENBANK® Accession No. NP_001161810.1, Oct. 6, 2024, all incorporated by reference. An exemplary amino acid sequence of a human plasmin protein is:

(SEQ ID NO: 2)
MEHKEVVLLLLLFLKSGQGEPLDDYVNTQGASLFSVTKKOLGAGSIEEC
AAKCEEDEEFTCRAFQYHSKEQQCVIMAENRKSSIIIRMRDVVLFEKKV
YLSECKTGNGKNYRGTMSKTKNGITCQKWSSTSPHRPRFSPATHPSEGL
EENYCRNPDNDPQGPWCYTTDPEKRYDYCDILECEEECMHCSGENYDGK
ISKTMSGLECQAWDSQSPHAHGYIPSKFPNKNLKKNYCRNPDRELRPWC
FTTDPNKRWELCDIPRCTTPPPSSGPTYQCLKGTGENYRGNVAVTVSGH
TCQHWSAQTPHTHNRTPENFPCKNLDENYCRNPDGKRAPWCHTTNSQVR
WEYCKIPSCDSSPVSTEQLAPTAPPELTPVVQDCYHGDGQSYRGTSSTT
TTGKKCQSWSSMTPHRHQKTPENYPNAGLTMNYCRNPDADKGPWCFTTD
PSVRWEYCNLKKCSGTEASVVAPPPVVLLPDVETPSEEDCMFGNGKGYR
GKRATTVTGTPCQDWAAQEPHRHSIFTPETNPRAGLEKNYCRNPDGDVG
GPWCYTTNPRKLYDYCDVPOCAAPSFDCGKPQVEPKKCPGRVVGGCVAH
PHSWPWQVSLRTRFGMHFCGGTLISPEWVLTAAHCLEKSPRPSSYKVIL
GAHQEVNLEPHVQEIEVSRLFLEPTRKDIALLKLSSPAVITDKVIPACL
PSPNYVVADRTECFITGWGETQGTFGAGLLKEAQLPVIENKVCNRYEFL
NGRVQSTELCAGHLAGGTDSCQGDSGGPLVCFEKDKYILQGVTSWGLGC
ARPNKPGVYVRVSRFVTWIEGVMRNN

Purified: A purified composition does not require absolute purity; rather, it is intended as a relative term. Thus, a purified population of cells is greater than about 90%, 91%, 92%, 93%, 94%, 95%, 96%, 97%, 98%, 99% or 100% pure, for example essentially free other cell types.

Retinal Ganglion Cell: A type of neuron located near the inner surface (the ganglion cell layer) of the retina of the eye. It receives visual information from photoreceptors via two intermediate neuron types: bipolar cells and retina amacrine cells. Retina amacrine cells, particularly narrow field cells, are important for creating functional subunits within the ganglion cell layer and making it so that ganglion cells can observe a small dot moving a small distance. RGCs collectively transmit image-forming and non-image-forming visual information from the retina in the form of action potential to several regions in the thalamus, hypothalamus, mesencephalon, or midbrain. The light passed through the eye is converted to electrical signals in photoreceptor cells, and the electrical signals produced are transmitted to RGCs via various neurons of the retina. RGCs function to receive these signals and transmit them to the optic nerve center of the brain. As part of the central nervous system, RGCs do not regenerate or are placed after injury in the mammalian. Thus, the cell replacement strategy provides an opportunity to restore RGCs.

Stem Cell: A cell that under suitable conditions is capable of differentiating into a diverse range of specialized cell types, while under other suitable conditions is capable of self-renewing and remaining in an essentially undifferentiated pluripotent state. The term “stem cell” also encompasses a pluripotent stem cell, multipotent stem cell, precursor cell and progenitor cell. Exemplary human stem cells can be obtained from hematopoietic or mesenchymal stem cells obtained from bone marrow tissue, embryonic stem cells obtained from embryonic tissue, or embryonic germ cells obtained from genital tissue of a fetus. Exemplary pluripotent stem cells can also be produced from somatic cells by reprogramming them to a pluripotent state by the expression of certain transcription factors associated with pluripotency; these cells are called “induced pluripotent stem cells” or “iPSCs”.

An “embryonic stem (ES) cell” is an undifferentiated pluripotent cell which is obtained from an embryo in an early stage, such as the inner cell mass at the blastocyst stage, or produced by artificial means (e.g., nuclear transfer) and can give rise to any differentiated cell type in an embryo or an adult, including germ cells (e.g., sperm and eggs).

“Induced pluripotent stem cells (iPSCs)” are cells generated by reprogramming a somatic cell by expressing or inducing expression of a combination of factors (herein referred to as reprogramming factors). iPSCs can be generated using fetal, postnatal, newborn, juvenile, or adult somatic cells. In certain aspects, factors that can be used to reprogram somatic cells to pluripotent stem cells include, for example, Oct4 (sometimes referred to as Oct 3/4), Sox2, c-Myc, and Klf4, Nanog, and Lin28. In some aspects, somatic cells are reprogrammed by expressing at least two reprogramming factors, at least three reprogramming factors, or four reprogramming factors to reprogram a somatic cell to a pluripotent stem cell.

The term “pluripotent” refers to the property of a cell to differentiate into all other cell types in an organism, with the exception of extraembryonic, or placental, cells. Pluripotent stem cells are capable of differentiating to cell types of all three germ layers (e.g., ectodermal, mesodermal, and endodermal cell types) even after prolonged culture. A pluripotent stem cell is an embryonic stem cell derived from the inner cell mass of a blastocyst. In other aspects, the pluripotent stem cell is an induced pluripotent stem cell derived by reprogramming somatic cells.

Subject: Human and non-human animals, including all vertebrates, such as mammals and non-mammals, such as non-human primates, mice, rabbits, sheep, dogs, cats, horses, cows, chickens, amphibians, and reptiles. In many aspects of the described methods, the subject is a human.

Therapeutically effective amount/effective amount: A quantity of a specified composition, pharmaceutical or therapeutic agent (such as plasmin, plasminogen, or ocriplasmin) sufficient to achieve a desired effect in a subject being treated with the agent. The effective amount of the agent will be dependent on several factors, including, but not limited to the subject being treated, the disease or condition being treated, and the manner of administration of the therapeutic composition. In some aspects of the present disclosure, the therapeutically effective amount (or effective amount) of plasmin, plasminogen or ocriplasmin is the amount required to degrade the ILM. A therapeutically effective amount (or effective amount) can be the amount needed to degrade the ILM, and, in combination with RGCs, treat glaucoma and/or increase retinal ganglion cell survival.

Transplantation of Cells: A procedure involving the transfer of exogenous cells into a site in a subject such that they can integrate into a tissue of interest. The exogenous cells can be autologous or allogeneic. RGC transplantation is the delivery of autologous or allogeneic retinal ganglion cells into the vitreous, onto the retinal surface, or into the ganglion cell layer, of a recipient eye.

Treating, Treatment, and Therapy: Any success or indicia of success in the attenuation or amelioration of an injury, pathology or condition, including any objective or subjective parameter such as abatement, remission, diminishing of symptoms or making the condition more tolerable to the patient, slowing in the rate of degeneration or decline, making the final point of degeneration less debilitating, improving a subject's physical or mental well-being, or improving vision. The treatment may be assessed by objective or subjective parameters; including the results of a physical examination, neurological examination, or psychiatric evaluations.

### Overview

Methods are disclosed herein for increasing integration of transplanted RGCs in a retina of a subject. These methods include administering an effective amount of ocriplasmin, plasmin, or plasminogen to an eye of the subject; and transplanting an effective amount of RGCs into the eye of the subject, thereby increasing the integration of RGCs in the retina in the eye of the subject.

Methods are also disclosed for treating glaucoma or optic neuropathy in an eye of a subject thereof. These methods include administering an effective amount of ocriplasmin, plasmin, or plasminogen to the eye of the subject; and transplanting an effective amount of RGCs into the eye of the subject, thereby treating the glaucoma or optic neuropathy in the eye of a subject.

In some aspects, the subject is human, and the retinal ganglion cells are human cells. In more aspects, the subject has optic neuropathy. In further aspects, the subject has glaucoma, such as normo-tensive glaucoma.

In some aspects, the method include administering an effective amount of an agent that lowers intraocular pressure to the eye of the subject. In aspects, the agent that lowers intraocular pressure is a) a prostaglandin analog, b) a beta-adrenergic blocker, c) an alpha-adrenergic agonist, or d) a cholinergic agonist. In non-limiting examples, the agent that lowers intraocular pressure is a) latanoprost, b) timolol, c) brimonidine, or d) pilocarpine.

In some aspects, the retinal ganglion cells are produced from stem cells in vitro prior to transplanting the retinal ganglion cells. In more aspects, the stem cells are induced pluripotent stem cells. In non-limiting examples, the stem cells are autologous.

In aspects, the method includes administering to the subject the effective amount of plasmin. The method can include administering to the subject a) about 3 internal international units (IU) to about 7 IU of plasmin; b) about 4 to about 6 IU of plasmin; or c) about 5 IU of plasmin.

In more aspects, the method includes administering the effective amount of ocriplasmin, plasmin, or plasminogen to the eye of the subject about 1 to about 3 weeks prior to transplanting the effective amount of retinal ganglion cells to the eye of the subject. In more aspects, the effective amount of ocriplasmin, plasmin, or plasminogen to the eye of the subject about 2 weeks prior to transplanting the effective amount of retinal ganglion cells to the eye of the subject.

In aspects, the retinal ganglion cells are in an organoid.

In some aspects, the effective amount of ocriplasmin, plasmin, or plasminogen is administered via intravitreal injection to the eye of the subject.

In more aspects, the method increases the survival of the retinal ganglion cells in the subject.

In aspects, the methods can include administering to the subject an effective amount of an immunosuppressive agent.

In further aspects, administering an effective amount of ocriplasmin, plasmin, or plasminogen to an eye of the subject comprises intravitral administration. In more aspects, transplanting the effective amount of retinal ganglion cells to the eye of the subject comprises intravitreal administration. In some aspects, administering an effective amount of ocriplasmin, plasmin, or plasminogen to an eye of the subject comprises intravitral administration, and transplanting the effective amount of retinal ganglion cells to the eye of the subject comprises intravitreal administration.

### Retinal Ganglion Cells (RGCs)

Methods of making RGCs from embryonic stem cells or iPSCs are known in the art, see for example, U.S. Pat. No. 11,168,830. For use in the therapeutic compositions, differentiation of mature RGCs from stem cells, for example, human embryonic stem cells or induced pluripotent stem cells, is induced in vitro, mature RGCs are proliferated and differentiated in a large amount and then administered to a patient having the above disease. The therapeutic composition may be formulated into dosage forms general in the art, for example, injectable formulations. The composition may be directly transplanted into a retinal site using a surgical procedure or may be intravitreally injected and migrate to a retinal site.

The therapeutic composition may further include an immunosuppressant to suppress immune rejection responses to grafts. The composition may further include a pharmaceutically acceptable carrier. The administration dose of the RGCs may vary depending on the severity of the patient, the route, method, and frequency of administration, the time period of treatment, the patient's age, sex, and disease severity, and may be easily determined by those skilled in the art according to various factors well-known in the medical art.

Still another aspect provides a method of treating glaucoma or optic neuropathy, including the step of administering the mature retinal ganglion to a subject suspected of having glaucoma or optic neuropathy, following degradation of the ILM by plasmin, plasminogen, or ocriplasmin.

The mature RGCs can be administered to any animal, and the animal may include livestock, such as cows, pigs, sheep, horses, dogs, mice, rats, cats, etc., as well as humans and primates. In aspects, wherein the stem cells are autologous, and thus the RGCs are autologous.

In other aspects, the stem cells are allogeneic, and thus the RGCs are allogeneic. The method can include administration of an immunosuppressive agent. RGCs can be human leukocyte antigen (HLA)-matched to a subject.

In aspects, the method includes administering to the subject the effective amount of plasmin. The method can include administering to the subject a) about 3 internal international units (IU) to about 7 IU of plasmin; b) about 4 to about 6 IU of plasmin; or c) about 5 IU of plasmin. The method can include administering the effective amount of ocriplasmin, plasmin, or plasminogen to the eye of the subject about 1 to about 3 weeks prior to transplanting the effective amount of RGCs to the eye of the subject, such as about 1, about 2 or about 3 weeks prior to transplanting the effective amount of RGCs. In aspects, the method includes administering the effective amount of ocriplasmin, plasmin, or plasminogen to the eye of the subject about 2 weeks prior to transplanting the effective amount of RGCs to the eye of the subject. In more aspects, the RGCs are in an organoid. In further aspects, the effective amount of ocriplasmin, plasmin, or plasminogen are administered via injection into the vitreous of the eye of the subject. In more aspects, the method increases the survival of the RGCs in the subject.

### Methods for Treating Glaucoma and Optic Neuropathy

Methods are disclosed herein for treating a subject with glaucoma. The subject can have open angle glaucoma, closed angle glaucoma, or normotensive glaucoma. The glaucoma can be a primary glaucoma or a secondary glaucoma. Methods are also disclosed for treating a subject with optic neuropathy. Any of these subjects can be selected for treatment. Methods are also disclosed for increasing integration of transplanted retinal ganglion cells in a retina of a subject.

These methods include administering an effective amount of ocriplasmin, plasmin, or plasminogen to an eye of the subject; and transplanting an effective amount of retinal ganglion cells into the eye of the subject, thereby increasing the integration of retinal ganglion cells in the retina in the eye of the subject.

The subject can be a veterinary subject or a human. The subject can be a mammal. The subject can be avian or a domestic pet, such as a cat, dog or rabbit. The subject can be a non-human primate, or livestock, including swine, ruminants, horses, and poultry.

With regard to glaucoma, intraocular pressure (IOP), the fluid pressure within the eye, can be measured in units of millimeters of mercury (mmHg) or kilopascals (kPa). Normal intraocular pressure is typically considered to be between 10 mmHg and 20 mmHg. The average value of intraocular pressure is 15.5 mmHg with fluctuations of about 2.75-3.50 mmHg. Elevated intraocular pressure (above 21 mmHg or 2.8 kPa) is the most important and only modifiable risk factor for glaucoma. In some aspects a subject is selected that has elevated intraocular pressure. In other aspects a subject is selected who has less than elevated intraocular pressure, but who has evidence of glaucomatous damage. For example, the subject may have cupping of the optic disc and an increased or increasing cup-to-disk ratio (for example greater than 0.3, 0.5 or 0.7). In other aspects the subject may have a slightly elevated IOP in the presence of glaucomatous optic nerve damage (such as a progression in the cup-to-disc ratio).

Testing for glaucoma can include measurements of the intraocular pressure, such as using tonometry, anterior chamber angle examination or gonioscopy, and examination of the optic nerve to identify damage, change in the cup-to-disc ratio, rim appearance and detection of vascular changes. Visual field testing can be performed. The retinal nerve fiber layer can be assessed with imaging techniques such as optical coherence tomography, scanning laser polarimetry, and/or scanning laser ophthalmoscopy (Heidelberg retinal tomogram). Additional tests include tonometry, ophthalmoscopy, perimetry, gonioscopy, pachymetry, and nerve fiber analysis. These methods can be performed in order to select a subject for treatment according to the methods disclosed herein.

In some aspects, the disclosed methods include selecting a subject with glaucoma, and administering to the subject a therapeutically effective amount of plasmin, plasminogen, or ocriplasmin. The disclosed methods also include administering a therapeutically effective amount of RGCs to the subject. In more aspects, the disclosed methods include selecting a subject with optic neuropathy, and administering to the subject a therapeutically effective amount of plasmin, plasminogen, or ocriplasmin. The disclosed methods also include administering a therapeutically effective amount of RGCs to the subject. The administration of the RGCs can occur about 1 to about 3 weeks following degradation of the ILM by the administration of the effective amount of ocriplasmin, plasmin or plasminogen, such as about 1, about 2 or about 3 weeks.

Administration can be to the vitreous of the eye using intravitreal injection. In some aspects, a therapeutically effective amount of an agent disclosed herein is administered by intraocular, for example intravitreal, injection. A general method for intravitreal injection may be illustrated by the following brief outline. This example is merely meant to illustrate certain features of the method, and is in no way meant to be limiting. Procedures for intravitreal injection are known in the art (see, for example Peyman, et al. (2009) Retina 29(7):875-912 and Fagan and Al-Qureshi, (2013) Clin. Experiment. Ophthalmol. 41(5):500-7). Other methods of intraocular administration are known in the art, and include subretinal administration.

Briefly, a subject for intravitreal injection may be prepared for the procedure by pupillary dilation, sterilization of the eye, and administration of anesthetic. Any suitable mydriatic agent known in the art may be used for pupillary dilation. Adequate pupillary dilation may be confirmed before treatment. Sterilization may be achieved by applying a sterilizing eye treatment, e.g., an iodide-containing solution such as povidone-iodine (BETADINE®). A similar solution may also be used to clean the eyelid, eyelashes, and any other nearby tissues (e.g., skin). Any suitable anesthetic may be used, such as lidocaine or proparacaine, at any suitable concentration. Anesthetic may be administered by any method known in the art, including without limitation topical drops, gels or jellies, and subconjunctival application of anesthetic.

Prior to injection, a sterilized eyelid speculum may be used to clear the eyelashes from the area. The site of the injection may be marked with a syringe. The site of the injection may be chosen based on the lens of the patient. For example, the injection site may be 3-3.5 mm from the limbus in pseudophakic or aphakic patients, and 3.5-4 mm from the limbus in phakic patients. The patient may look in a direction opposite the injection site. During injection, the needle can be inserted perpendicular to the sclera and pointed to the center of the eye. The needle can be inserted such that the tip ends in the vitreous, rather than the subretinal space. Any suitable volume known in the art for injection may be used. After injection, the eye can be treated with a sterilizing agent such as an antibiotic. The eye can also be rinsed to remove excess sterilizing agent.

Intravitreal injection of a therapeutic agent, such as plasmin, plasminogen, or ocriplasmin, can be performed once, or can be performed repeatedly, such as 2 or more times.

The subject can be administered additional therapeutic agents. For the treatment of glaucoma, these include, but are not limited to, an agent that lowers intraocular pressure. The agent can be a) a prostaglandin analog, b) a beta-adrenergic blocker, c) an alpha-adrenergic agonist, or d) a cholinergic agonist. Exemplary agents include latanoprost, bimatorpost, travoprost, timolol, betaxolol, brimonidine, pilocarpine, dorzolamide, brinzolamide, and acetazolamide. In some specific non-limiting examples, the agent is a) latanoprost, b) timolol, c) brimonidine, or d) pilocarpine. The subject can be administered a Rho-kinase inhibitor, such as, but not limited to, ripasudil or netarsudil.

Additional agents that can be administered to the subject include antibacterial and antifungal antibiotics, as well as non-steroidal anti-inflammatory agents to reduce risk of infection and inflammation. Additional agents can be administered by any route. The additional agents can be formulated separately, or in the same composition.

Agents of use include minoglycosides (for example, amikacin, apramycin, arbekacin, bambermycins, butirosin, dibekacin, dihydrostreptomycin, fortimicin(s), gentamicin, isepamicin, kanamycin, micronomicin, neomycin, neomycin undecylenate, netilmicin, paromomycin, ribostamycin, sisomicin, spectinomycin, streptomycin, tobramycin, trospectomycin), amphenicols (for example, azidamfenicol, chloramphenicol, florfenicol, thiamphenicol), ansamycins (for example, rifamide, rifampin, rifamycin sv, rifapentine, rifaximin), β-lactams (for example, carbacephems (e.g., loracarbef), carbapenems (for example, biapenem, imipenem, meropenem, panipenem), cephalosporins (for example, cefaclor, cefadroxil, cefamandole, cefatrizine, cefazedone, cefazolin, cefcapene pivoxil, cefclidin, cefdinir, cefditoren, cefepime, cefetamet, cefixime, cefmenoxime, cefodizime, cefonicid, cefoperazone, ceforanide, cefotaxime, cefotiam, cefozopran, cefpimizole, cefpiramide, cefpirome, cefpodoxime proxetil, cefprozil, cefroxadine, cefsulodin, ceftazidime, cefteram, ceftezole, ceftibuten, ceftizoxime, ceftriaxone, cefuroxime, cefuzonam, cephacetrile sodium, cephalexin, cephaloglycin, cephaloridine, cephalosporin, cephalothin, cephapirin sodium, cephradine, pivcefalexin), cephamycins (for example, cefbuperazone, cefmetazole, cefininox, cefotetan, cefoxitin), monobactams (for example, aztreonam, carumonam, tigemonam), oxacephems, flomoxef, moxalactam), penicillins (for example, amdinocillin, amdinocillin pivoxil, amoxicillin, ampicillin, apalcillin, aspoxicillin, azidocillin, azlocillin, bacampicillin, benzylpenicillinic acid, benzylpenicillin sodium, carbenicillin, carindacillin, clometocillin, cloxacillin, cyclacillin, dicloxacillin, epicillin, fenbenicillin, floxacillin, hetacillin, lenampicillin, metampicillin, methicillin sodium, mezlocillin, nafcillin sodium, oxacillin, penamecillin, penethamate hydriodide, penicillin G benethamine, penicillin g benzathine, penicillin g benzhydrylamine, penicillin G calcium, penicillin G hydrabamine, penicillin G potassium, penicillin G procaine, penicillin N, penicillin O, penicillin V, penicillin V benzathine, penicillin V hydrabamine, penimepicycline, phenethicillin potassium, piperacillin, pivampicillin, propicillin, quinacillin, sulbenicillin, sultamicillin, talampicillin, temocillin, ticarcillin), other (for example, ritipenem), lincosamides (for example, clindamycin, lincomycin), macrolides (for example, azithromycin, carbomycin, clarithromycin, dirithromycin, erythromycin, erythromycin acistrate, erythromycin estolate, erythromycin glucoheptonate, erythromycin lactobionate, erythromycin propionate, erythromycin stearate, josamycin, leucomycins, midecamycins, miokamycin, oleandomycin, primycin, rokitamycin, rosaramicin, roxithromycin, spiramycin, troleandomycin), polypeptides (for example, amphomycin, bacitracin, capreomycin, colistin, enduracidin, enviomycin, fusafungine, gramicidin s, gramicidin(s), mikamycin, polymyxin, pristinamycin, ristocetin, teicoplanin, thiostrepton, tuberactinomycin, tyrocidine, tyrothricin, vancomycin, viomycin, virginiamycin, zinc bacitracin), tetracyclines (for example, apicycline, chlortetracycline, clomocycline, demeclocycline, doxycycline, guamecycline, lymecycline, meclocycline, methacycline, minocycline, oxytetracycline, penimepicycline, pipacycline, rolitetracycline, sancycline, tetracycline), and others (e.g., cycloserine, mupirocin, tuberin). Agents of use also include synthetic antibacterials, such as 2,4-Diaminopyrimidines (for example, brodimoprim, tetroxoprim, trimethoprim), nitrofurans (for example, furaltadone, furazolium chloride, nifuradene, nifuratel, nifurfoline, nifurpirinol, nifurprazine, nifurtoinol, nitrofurantoin), quinolones and analogs (for example, cinoxacin, ciprofloxacin, clinafloxacin, difloxacin, enoxacin, fleroxacin, flumequine, grepafloxacin, lomefloxacin, miloxacin, nadifloxacin, nalidixic acid, norfloxacin, ofloxacin, oxolinic acid, pazufloxacin, pefloxacin, pipemidic acid, piromidic acid, rosoxacin, rufloxacin, sparfloxacin, temafloxacin, tosufloxacin, trovafloxacin), sulfonamides (for example, acetyl sulfamethoxypyrazine, benzylsulfamide, chloramine-b, chloramine-t, dichloramine t, mafenide, 4′-(methylsulfamoyl) sulfanilanilide, noprylsulfamide, phthalylsulfacetamide, phthalylsulfathiazole, salazosulfadimidine, succinylsulfathiazole, sulfabenzamide, sulfacetamide, sulfachlorpyridazine, sulfachrysoidine, sulfacytine, sulfadiazine, sulfadicramide, sulfadimethoxine, sulfadoxine, sulfaethidole, sulfaguanidine, sulfaguanol, sulfalene, sulfaloxic acid, sulfamerazine, sulfameter, sulfamethazine, sulfamethizole, sulfamethomidine, sulfamethoxazole, sulfamethoxypyridazine, sulfametrole, sulfamidocchrysoidine, sulfamoxole, sulfanilamide, sulfanilylurea, n-sulfanilyl-3,4-xylamide, sulfanitran, sulfaperine, sulfaphenazole, sulfaproxyline, sulfapyrazine, sulfapyridine, sulfasomizole, sulfasymazine, sulfathiazole, sulfathiourea, sulfatolamide, sulfisomidine, sulfisoxazole) sulfones (for example, acedapsone, acediasulfone, acetosulfone sodium, dapsone, diathymosulfone, glucosulfone sodium, solasulfone, succisulfone, sulfanilic acid, p-sulfanilylbenzylamine, sulfoxone sodium, thiazolsulfone), and others (for example, clofoctol, hexedine, methenamine, methenamine anhydromethylene-citrate, methenamine hippurate, methenamine mandelate, methenamine sulfosalicylate, nitroxoline, taurolidine, xibornol).

Additional agents of use include antifungal antibiotics such as polyenes (for example, amphotericin B, candicidin, dennostatin, filipin, fungichromin, hachimycin, hamycin, lucensomycin, mepartricin, natamycin, nystatin, pecilocin, perimycin), others (for example, azaserine, griseofulvin, oligomycins, neomycin undecylenate, pyrrolnitrin, siccanin, tubercidin, viridin) allylamines (for example, butenafine, naftifine, terbinafine), imidazoles (for example, bifonazole, butoconazole, chlordantoin, chlormiidazole, cloconazole, clotrimazole, econazole, enilconazole, fenticonazole, flutrimazole, isoconazole, ketoconazole, lanoconazole, miconazole, omoconazole, oxiconazole nitrate, sertaconazole, sulconazole, tioconazole), thiocarbamates (for example, tolciclate, tolindate, tolnaftate), triazoles (for example, fluconazole, itraconazole, saperconazole, terconazole) others (for example, acrisorcin, amorolfine, biphenamine, bromosalicylchloranilide, buclosamide, calcium propionate, chlorphenesin, ciclopirox, cloxyquin, coparaffinate, diamthazole dihydrochloride, exalamide, flucytosine, halethazole, hexetidine, loflucarban, nifuratel, potassium iodide, propionic acid, pyrithione, salicylanilide, sodium propionate, sulbentine, tenonitrozole, triacetin, ujothion, undecylenic acid, zinc propionate). Antineoplastic agents can also be of use including (1) antibiotics and analogs (for example, aclacinomycins, actinomycin, anthramycin, azaserine, bleomycins, cactinomycin, carubicin, carzinophilin, chromomycins, dactinomycin, daunorubicin, 6-diazo-5-oxo-L-norleucine, doxorubicin, epirubicin, idarubicin, menogaril, mitomycins, mycophenolic acid, nogalamycin, olivomycines, peplomycin, pirarubicin, plicamycin, porfiromycin, puromycin, streptonigrin, streptozocin, tubercidin, zinostatin, zorubicin), (2) antimetabolites such as folic acid analogs (for example, denopterin, edatrexate, methotrexate, piritrexim, pteropterin, trimetrexate), (3) purine analogs (for example, cladribine, fludarabine, 6-mercaptopurine, thiamiprine, thioguanine), (4) pyrimidine analogs (for example, ancitabine, azacitidine, 6-azauridine, carmofur, cytarabine, doxifluridine, emitefur, enocitabine, floxuridine, fluorouracil, gemcitabine, tagafur).

Steroidal anti-inflammatory agents can also be used such as 21-acetoxypregnenolone, alclometasone, algestone, amcinonide, beclomethasone, betamethasone, budesonide, chloroprednisone, clobetasol, clobetasone, clocortolone, cloprednol, corticosterone, cortisone, cortivazol, cyclosporine, deflazacort, desonide, desoximetasone, dexamethasone, diflorasone, diflucortolone, difluprednate, enoxolone, fluazacort, flucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, halopredone acetate, hydrocortamate, hydrocortisone, loteprednol etabonate, mazipredone, medrysone, meprednisone, methylprednisolone, mometasone furoate, paramethasone, prednicarbate, prednisolone, prednisolone 25-diethylamino-acetate, prednisolone sodium phosphate, prednisone, prednival, prednylidene, rimexolone, tixocortol, triamcinolone, triamcinolone acetonide, triamcinolone benetonide, and triamcinolone hexacetonide.

In addition, non-steroidal anti-inflammatory agents can be used. These include aminoarylcarboxylic acid derivatives (for example, enfenamic acid, etofenamate, flufenamic acid, isonixin, meclofenamic acid, mefenamic acid, niflumic acid, talniflumate, terofenamate, tolfenamic acid), arylacetic acid derivatives (for example, aceclofenac, acemetacin, alclofenac, amfenac, amtolmetin guacil, bromfenac, bufexamac, cinmetacin, clopirac, diclofenac sodium, etodolac, felbinac, fenclozic acid, fentiazac, glucametacin, ibufenac, indomethacin, isofezolac, isoxepac, lonazolac, metiazinic acid, mofezolac, oxametacine, pirazolac, proglumetacin, sulindac, tiaramide, tolmetin, tropesin, zomepirac), arylbutyric acid derivatives (for example, bumadizon, butibufen, fenbufen, xenbucin), arylcarboxylic acids (for example, clidanac, ketorolac, tinoridine), arylpropionic acid derivatives (for example, alminoprofen, benoxaprofen, bermoprofen, bucloxic acid, carprofen, fenoprofen, flunoxaprofen, flurbiprofen, ibuprofen, ibuproxam, indoprofen, ketoprofen, loxoprofen, naproxen, oxaprozin, piketoprolen, pirprofen, pranoprofen, protizinic acid, suprofen, tiaprofenic acid, ximoprofen, zaltoprofen), pyrazoles (for example, difenamizole, epirizole), pyrazolones (for example, apazone, benzpiperylon, feprazone, mofebutazone, morazone, oxyphenbutazone, phenylbutazone, pipebuzone, propyphenazone, ramifenazone, suxibuzone, thiazolinobutazone), salicylic acid derivatives (for example, acetaminosalol, aspirin, benorylate, bromosaligenin, calcium acetylsalicylate, diflunisal, etersalate, fendosal, gentisic acid, glycol salicylate, imidazole salicylate, lysine acetylsalicylate, mesalamine, morpholine salicylate, 1-naphthyl salicylate, olsalazine, parsalmide, phenyl acetylsalicylate, phenyl salicylate, salacetamide, salicylamide o-acetic acid, salicylsulfuric acid, salsalate, sulfasalazine), thiazinecarboxamides (for example, ampiroxicam, droxicam, isoxicam, lornoxicam, piroxicam, tenoxicam), .epsilon.-acetamidocaproic acid, s-adenosylmethionine, 3-amino-4-hydroxybutyric acid, amixetrine, bendazac, benzydamine, .alpha.-bisabolol, bucolome, difenpiramide, ditazol, emorfazone, fepradinol, guaiazulene, nabumetone, nimesulide, oxaceprol, paranyline, perisoxal, proquazone, superoxide dismutase, tenidap, and zileuton.

Immunosuppressive agents of use include, but are not limited to steroids, calcineurin inhibitors, monoclonal antibodies and polyclonal antibodies. The steroid can be a corticosteroid. Examples of corticosteroids include but are not limited to prednisone and methylprednisone. Examples of calcineurin inhibitors include but are not limited to cyclosporine and tacrolimus. Examples of monoclonal antibodies include but are not limited to anti-CD3 antibody (Muromonab-CD3; Orthoclone OKT3®) and anti-IL-2R alpha chain (p55). Examples of polyclonal antibodies include but are not limited to anti-thymocyte globulin-equine (ATGAM®) and anti-thymocyte globulin-rabbit (RATG thymoglobuline). Immunosuppressant drugs include, but are not limited to, corticosteroids (e.g., prednisone), calcineurin inhibitors (e.g., tacrolimus, cyclosporine), inosine monophosphate dehydrogenase (IMDH) inhibitors (e.g., mycophenolate mofetil), Janus kinase inhibitors (e.g., XELIJANZ®), mechanistic target of rapamycin (mTOR) inhibitors (e.g., sirolimus), anti-metabolites (e.g., mycophenolate mofetil (MMF) or mycophenolic acid (MPA)), and biologics (e.g., basiliximab, adalimumab, infliximab, alemtuzumab, belatacept).

Following administration, the subject can be evaluated for response using any methods known in the art. These include, but are not limited to, optical coherence tomography, ophthalmoscopy, perimetry, gonioscopy, pachymetry, or nerve fiber analysis. In some aspects, retinal ganglion cell number and/or viability can be assessed. One of skill in the art can readily determine that the disclosed methods are effective. For example it can be determined by whether the cup-to-disc ratio has stabilized. Scanning laser polarimetry or optical coherence tomography could be used, for example to perform retinal nerve fiber layer analysis. A visual field test could be used to monitor progression of glaucoma. For any of the disclosed methods, therapeutic efficacy in treating a vision deficiency can as an alteration in the individual's vision.

### EXAMPLES

RGCs are the sole conduit for transmitting visual information from the eye to the brain. In mammals, RGCs lack the ability to regenerate after injury, making cell replacement a potential therapeutic strategy for optic neuropathies. However, the ILM serves as a major barrier, hindering donor cell integration.

A significant challenge encountered when transplanting cells into the host eye is the presence of the ILM, a basement membrane primarily composed of extracellular matrix proteins like laminin and collagen IV, which acts as a significant barrier (Johnson et al., Ophthalmol Sci. December 2023; 3(4):100390; Soucy et al., Mol Neurodegener. Sep. 21, 2023; 18(1):64). This barrier obstructs the migration of transplanted stem cells, viral vectors, therapeutic compounds, and nanoparticles, thereby limiting the engraftment of RGCs (Aguzzi et al., bioRxiv. 2022:2022.2012.2013.519327; Johnson et al., Invest Ophthalmol Vis Sci. February 2010; 51(2):960-970; Peynshaert et al., Curr Eye Res. May 2019; 44(5):465-475).

The efficacy and immune response of plasmin were examined in a rodent model. Plasmin is a vitreolytic enzyme analogous to the clinically used ocriplasmin. Ocriplasmin, marketed under the brand name JETREA®, is a recombinant protease that targets fibronectin and laminin, which are integral components of the vitreoretinal interface.

Although ocriplasmin was discontinued in 2020, plasmin—a non-specific serine protease with fibrinolytic properties—remains a key enzyme with similar biological activity. In addition to its role in fibrinolysis, plasmin can hydrolyze various glycoproteins, including laminin and fibronectin.

These findings identify an efficient plasmin treatment dose that effectively disrupts the ILM without inducing immune cell infiltration, microglial reactivity or damage to RGCs. Furthermore, a method is provided to enhance donor RGCs survival following transplantation by ILM disruption, without inducing ocular inflammation.

### Example 1

### Materials and Methods

Cryosection immunostaining: For the preparation of retinal cryosections, mouse eye globes were incubated in 4% PFA at 4° C. overnight for fixation. Subsequently, they were washed 3 times (15 minutes each) with PBS and sequentially incubated in 15% sucrose, followed by 30% sucrose both at 4° C. overnight. After, they were embedded in Optimal Cutting Temperature (Fisher scientific, 23-730-571) mounting medium and cryosections of 12 μm thickness were cut. Sections were permeabilized with 0.1% Triton X-100 (Sigma Aldrich, X100) in PBS for 30 minutes and blocked with 5% normal goat serum (NGS) (Invitrogen, 10000C) and 0.1% Triton X-100 in PBS for 1 hour at room temperature. The sections were incubated overnight at 4° C. with different primary antibodies such as mouse anti-Brn3a (1:100, Millipore Sigma, MAB1585), rabbit anti-laminin antibody (1:100, PA1-16730, Thermo Scientific) or rabbit anti-Iba1 (1:100, 019-19741, Fujifilm). Following 3 washes with PBS (15 minutes each) sections were incubated overnight at 4° C. with nuclear stain 4′,6-diamidino-2-phenylindole (DAPI) (1:5000, Sigma Aldrich, D9542) and secondary antibodies Alexa Fluor 488 goat anti-mouse (1:500, #A32723, Invitrogen), Alexa Fluor 555 goat anti-rabbit (1:500, #A32732, Invitrogen) or Alexa Fluor 488 goat anti-rabbit (1:500, #A-11034, Invitrogen). Finally, imaging was conducted using an Olympus Life Science IX83 inverted microscope.

Retina flatmount immunostaining: To create the retina flatmount, following dissection and fixation in 4% PFA for 1 hour, mouse retinas were flattened with four relaxing cuts dividing them into four quadrants. Blocking was performed using 5% normal goat serum (NGS) and 1% triton X-100 in PBS for 1 hour. Furthermore, retinas were incubated overnight at 4° C. with the mouse anti-Brn3a (1:100, Millipore Sigma, MAB1585) or the rabbit anti-RBPMS (1:100, Sigma Aldrich, ABN1362). After undergoing three washes with PBS of 15 minutes each, the retina flatmount samples were incubated overnight with ALEXAFLUOR™ 488 goat anti-mouse secondary antibody (1:500, #A32723, Invitrogen) or ALEXAFLUOR™ 488 goat anti-rabbit (1:500, #A-11034, Invitrogen) along with DAPI (1:500, #D9542, Sigma-Aldrich). The samples were then washed three more times for 15 minutes each in PBS and then the retinas were covered with 1.5-mm coverslips using anti-fade mounting medium (ProLong Gold, Life Technologies). Imaging of the samples was conducted using the Leica Microsystems DMi8 automated inverted microscope, which has the ability to spirally scan slides and take photos of the whole flatmount retina. Olympus Life Science IX83 confocal inverted microscope was used to create z-stack images.

Histopathology: To evaluate the immune infiltration in the vitreous cavity 8- to 10-week-old mice were injected with PBS or plasmin (2.3 μg or 4.6 μg) (EMD Millipore, 527621-10U). Two weeks later, the mice were euthanized, and their eyes were enucleated and fixed in Davidson's (Hartmann's) Fixative (Electron Microscopy Sciences, 64133-10) at room temperature for 24 hours. The ocular globes were then embedded in paraffin, and 5-μm sections were cut and stained with hematoxylin and eosin. The vitreous and retina were examined under light microscopy.

For the LPS-induced uveitis reference picture, 8- to 10-week-old mice were intravitreally injected with E. coli lipopolysaccharide O111:B4 (250 ng in 2 μl; Sigma Aldrich, L2630). The mice were euthanized 24 hours after injection, and their eyes were enucleated and fixed in Davidson's (Hartmann's) Fixative at room temperature for 24 hours. The ocular globes were then embedded in paraffin, and 5-μm sections were sectioned and stained with hematoxylin and eosin.

Human retinal organoid differentiation: Human retinal organoids were differentiated from a transgenic human embryonic stem cell line Brn3b-tdTomato-Thy1.2 following lab protocol (Rao et al., Methods Mol Biol. 2025; 2848:187-196).

Briefly, stem cells maintained in StemFlex medium (Gibco, #A3349401) were passaged by adding 0.5 mM trypsin-ethylenediaminetetraacetic acid (EDTA) (Gibco, 25-300-120) to the culture medium for 5 minutes at 37° C. and resuspended in a 3:1 ratio of StemFlex and neural induction medium (NIM) on Day 0 to initiate embryonic body (EBs) formation. NIM consisted of 1× N2 supplement (100×, Gibco, 17502-048), 1×MEM non-essential amino acids solution (100×, Gibco, 11140050), 2 μg/mL Heparin (0.2%, Stemcell Technologies, 07980) and 1× Antibiotic-Antimycotic (100×, Gibco, 15240062) in DMEM/F12 (1:1, Gibco, 11-330-032). On Day 1, the EBs were transferred to a 1:1 ratio of StemFlex and NIM medium, followed by a 1:3 ratio of StemFlex and NIM medium on Day 2. On Day 3, the EBs were resuspended in NIM and seeded on Matrigel-coated wells (Corning, CLS354277), with media changed occurring every other day.

After 16 days of differentiation, the medium was changed to Retinal Differentiation Medium (RDM), consisting of 57.6% DMEM/F12 (1:1), 38.4% DMEM (Gibco, 11885084), 1×B-27™ Supplement (minus vitamin A, 50× Gibco, 12587010), 1×MEM non-essential amino acids solution, and 1× Antibiotic-Antimycotic (100×). On Day 28, the cells were gently scraped in a single 360° motion using a cell scraper and transferred to a 6-well flat-bottom ultra-low attachment dish (Genesee Scientific, #25-105). The cell medium was switched to RC2, composed of 50% DMEM/F12, 35% DMEM basic, 1×B-27™ Supplement, 10% Fetal Bovine Serum (Gibco, 10437028), 1×GlutaMax (100×, Gibco, 35050061), 100 μM Taurine (100 mM, Sigma, T-0625), 1×MEM non-essential amino acids solution, and 1× Antibiotic-Antimycotic (100×). Additionally, 1 μM Retinoic acid (Sigma Aldrich, R2625) was supplemented every other day for 2 weeks after scraping to facilitate maturation. RC2 medium was changed every two days, and human retinal organoids were maintained in this medium until the indicated experimental time points.

Donor cell transplantation: 8- to 10-week-old C57BL/6J mice (either sex, Jackson Laboratory) were randomly separated into three groups (PBS, 2.3 μg and 4.6 μg of plasmin) and given ad libitum access to food and water. This research was conducted in compliance with the Association for Research in Vision and Ophthalmology Statement for the Use of Animals in Ophthalmic and Vision Research. Mice were anesthetized with ketamine/xylazine (40-55 mg/kg ketamine, 4-5.5 mg/kg xylazine). Proparacaine (0.5%, Sandoz, PO-6543) as a local anesthetic was also applied topically to the cornea. The non-operated eye was kept moist with GENTEAL® artificial tear drops (Alcon, NDC 0065-0426-36). 2.3 μg or 4.6 μg of plasmin (EMD Millipore, 527621-10U) or PBS was injected intravitreally into the mice eyes using a 30-gauge needle on a 5-μL glass Hamilton syringe. Care was taken to prevent damage to the lens and to ensure that the retinal blood supply remained unaffected. Two weeks after plasmin injection, each mouse received an intravitreal injection of 20,000 cells in 2 μl of medium. One week after transplantation, mice were sacrificed by a rising concentration of CO₂ and perfused/fixed intracardially with PBS and 4% paraformaldehyde (PFA). Eyes were surgically dissected, immersed in 4% PFA overnight at 4° C. and flatmounts were created and stained accordingly.

For cell transplantation, the hROs were dissociated with ACCUMAX™ solution (Sigma-aldrich, A7089) at 37° C. for 30 min. After incubation, the hRO derived-cells were centrifuged and resuspended with NB-SATO growth medium containing a Rho-kinase (ROCK) inhibitor (Sigma-Aldrich, Y-27632). Growth medium was made by first preparing SATO. SATO (100×) was made by adding 400 mg Transferrin (Sigma Aldrich, T-1147), 400 mg bovine serum albumin (BSA) (Sigma Aldrich, A-4161), 10 μl progesterone (2.5 mg/100 ml EtOH, Sigma Aldrich, P8783), 64 mg putrescine dihydrochloride (Sigma Aldrich, P7507), 400 μl sodium selenite (4 mg/100 μl 0.1N NaOH and 10 ml neurobasal, Sigma Aldrich, S5261). Consequently, it was filtered through 0.22 μm filter (Sigma Aldrich, SLGV013SL). The final NB-SATO growth medium was prepared by adding 200 μl Penicillin-Streptomycin (Gibco, 15140122), 200 μl insulin (5 μg/ml, Sigma Aldrich, I6634), 200 μl sodium pyruvate (100 mM, Gibco, 11360070), 200 μl triiodothyronine (T3) (100×, Sigma Aldrich, T6397), 200 μl 1 mM L-glutamine (100×, Gibco, A2916801), 400 μl B27 (50×, Gibco, 17-504-044), 200 μl SATO (100×) and 20 μl N-acetyl-L-cysteine (NAC) (5 mg/ml, 1000×) in 20 ml Neurobasal medium (Gibco, 21103049). NAC was made by dissolving 50 mg N-acetyl-L-cysteine (Sigma Aldrich, A8199) in 10 ml neurobasal medium. When all ingredients of NB-SATO were combined it was filtered through a 0.22 μm filter. After filtration 20 μl BDNF (1000×, 50 μg/ml, PeproTech, 450-02), 20 μl CNTF (1000×, 10 μg/ml, PeproTech, 450-13), forskolin (1000×, 5 mM, Sigma Aldrich, 6866) and 40 μl FGF (500×, 20 μg/ml, PeproTech, 100-18B) were added.

Electroretinography: The whiskers of 8- to 10-week-old C57BL/6 wild-type mice were trimmed, followed by an overnight adaptation period. They were then anesthetized with an intraperitoneal injection of a ketamine/xylazine mixture (100 mg/mL) at a dose of 5 mL/kg under dim red light. Pupils were fully dilated using 2.5% phenylephrine combined with 1% tropicamide eye drops and their cornea was anesthetized by 0.5% proparacaine drops. Following anesthesia, the mice were placed on a heated platform. GENTEAL® severe lubricant eye gel (Alcon, NDC 0078-0429-47) was applied to their eyes for lubrication. Electroretinograms were recorded with the Celeris system (Diagnosys, LLC). Pattern electroretinograms (pERGs) were recorded using one pattern stimulator along with one full field stimulator as the fellow eye reference electrode. Full-field electroretinograms (ffERG) were recorded using two full field simulators. The electrodes were lubricated with GenTeal to enhance conductance and placed in contact with each cornea and aligned with the center of the pupils. Before presenting the stimuli, electrode impedance was confirmed to be below 10 KΩ. Under this arrangement, in the pERG, stimuli were presented to one eye while the other, the unstimulated eye, acted as the reference. The pERG response was elicited by a contrast reversing bar stimulus with a spatial frequency of 0.059 cycles/degree, mean luminance of 50 cd/m², and reversal rate of 2.1/sec. A total of 400 sweeps were averaged for analysis. The full pERG amplitude was determined from the positive peak at P1 to the negative trough at N2, often termed as PIN2. pERG responses were recorded 2 weeks after plasmin injections, 1 month after plasmin injections and 2 weeks after 6-aminohexanoic acid injections (Sigma-Aldrich, 07260). In the ffERG, white flashes of 0.5 cd·s·m⁻² were delivered to elicit retinal responses. All readings were recorded using the Espion software from Diagnosys.

Optical Coherence Tomography: Optical coherence tomography (OCT) scans were obtained two weeks post-treatment. Imaging was conducted using a visible light OCT (vis-OCT) prototype developed at the UPMC Vision Institute. Retinal vis-OCT scans were acquired at an A-line sampling rate of 70 kHz. The system utilized a light spectrum ranging from 510 nm to 610 nm, generated by a laser (EXU-6 OCT; NKT Photonics, Boston, MA, USA), providing an axial resolution of 1.2 μm in tissue. The incident power on the cornea was maintained at 0.8 mW. Volumetric raster scans, consisting of 500×2×500 A-lines near the optic disc, were acquired for retinal analysis and RGC axon bundle assessment.

To ensure boundary accuracy, retinal layers were segmented automatically with manual correction. Retinal layer thickness measurements were performed to assess retinal degeneration following treatment, specifically ganglion cell complex (GCC) between the ILM and the inner plexiform layer (IPL), as well as whole retina between the ILM and Bruch's membrane (BM). Data were categorized into three experimental groups: eyes treated with phosphate-buffered saline (PBS), eyes treated with 2.3 μg of plasmin, and eyes treated with 4.6 μg of plasmin.

Quantification and statistical analysis: The number of mice used in each experiment is indicated in the figures and/or the figure legends. Linear percentage coverage of laminin was calculated by measuring the average of laminin coverage at 20× magnification from three fields of view from three sections for each eye. Cell counting for Iba1+ and Brn3a+ was conducted from one field of view from one section. For retinal wholemounts, each retina was divided into four quadrants. In each quadrant, Brn3a⁺ cells were counted from a single randomly selected digital micrograph taken from the peripheral region, located 1.5 mm from the optic nerve head. TdTomato+ cells after transplantation were counted manually from the whole retina of each sample. Quantification of cell numbers measured in retinal sections was normalized to liner micrometer of retinal cell layer, using central sections near the optic nerve head as a standard to select slides for cell counting. Cells were manually counted in a masked manner. Statistical analysis was performed by calculating the mean±SEM. Unpaired t-tests and one-way ANOVA followed by post hoc t-tests with Tukey's correction was conducted for data analysis. P<0.05 was considered as significant. Graphs were created using Prism 10 software (GraphPad, La Jolla, CA, USA).

### Example 2

### Intravitreal Administration of the Optimal Plasmin Dose does not Cause Immune Cell Infiltration or Microglia Activation

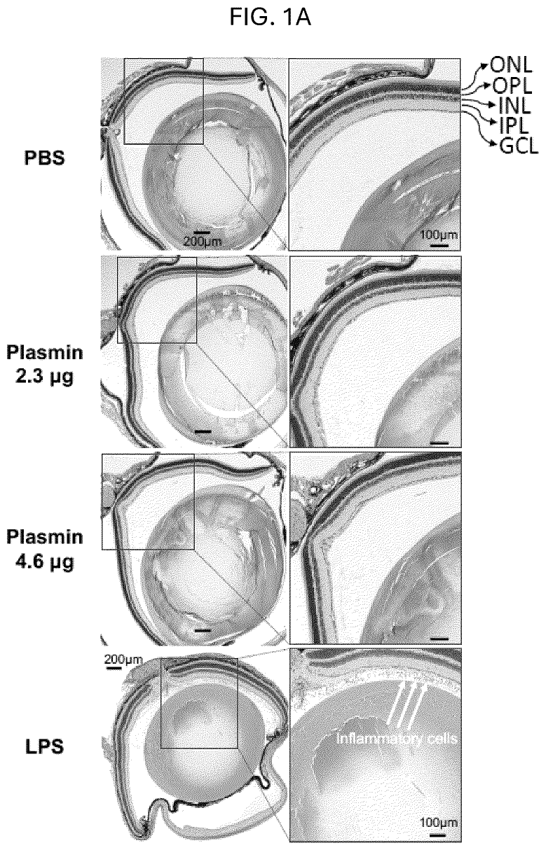

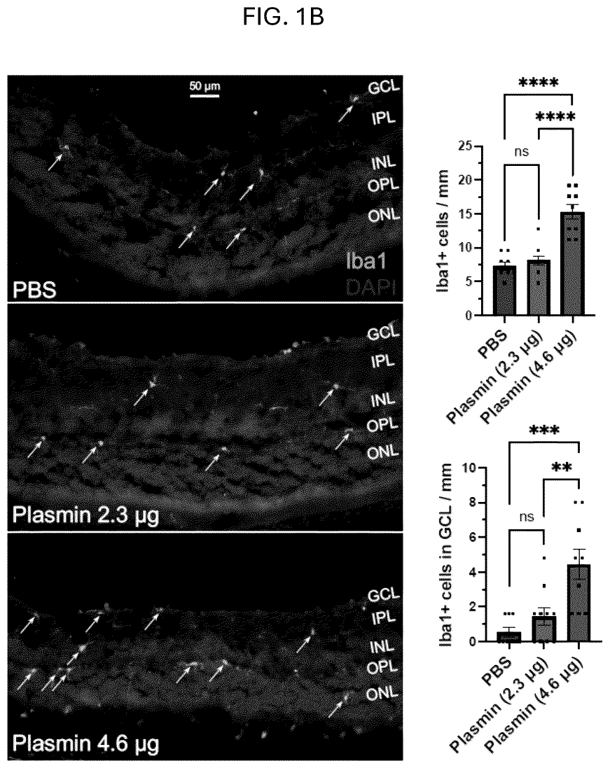

In a pig model, treatment with ocriplasmin did not induce inflammatory responses (Jonckx et al., J Ophthalmol. 2017; 2017:2060765). To determine whether this holds true in mice, mice were intravitreally injected with phosphate-buffered saline (PBS), 2.3 μg, or 4.6 μg of plasmin. Two weeks post-treatment, histological examination of the eyes revealed no immune cell infiltration in either the 2.3 or 4.6 μg groups (FIG. 1A), confirming that plasmin treatment did not trigger inflammatory responses in the vitreous cavity

The impact on resident retinal immune cells was further investigated; specifically microglia, by preparing cryosections of PBS-, 2.3 μg-, and 4.6 μg-treated retinas and staining them with the microglia marker Ionized Calcium-Binding Adaptor Molecule 1 (Iba1). While no significant difference was observed between the PBS and 2.3 μg groups, the 4.6 μg group showed a notable increase in microglia, particularly in the ganglion cell layer (FIG. 1B). These findings suggest that the 2.3 μg dose is better tolerated, as it mitigates the microglial reactivity observed at the 4.6 μg dose, making it a more suitable candidate for further experiments.

### Example 3

### Plasmin Treatment is Capable of Disrupting the Internal Limiting Membrane without Damaging RGCs

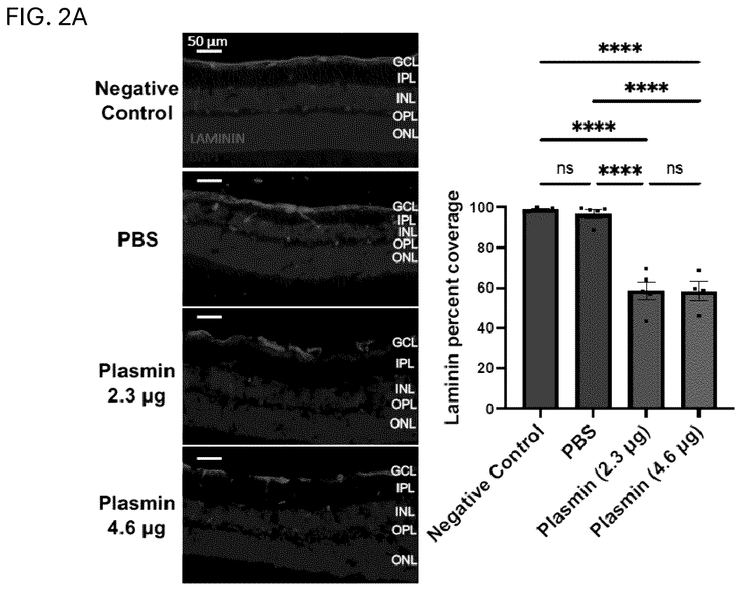

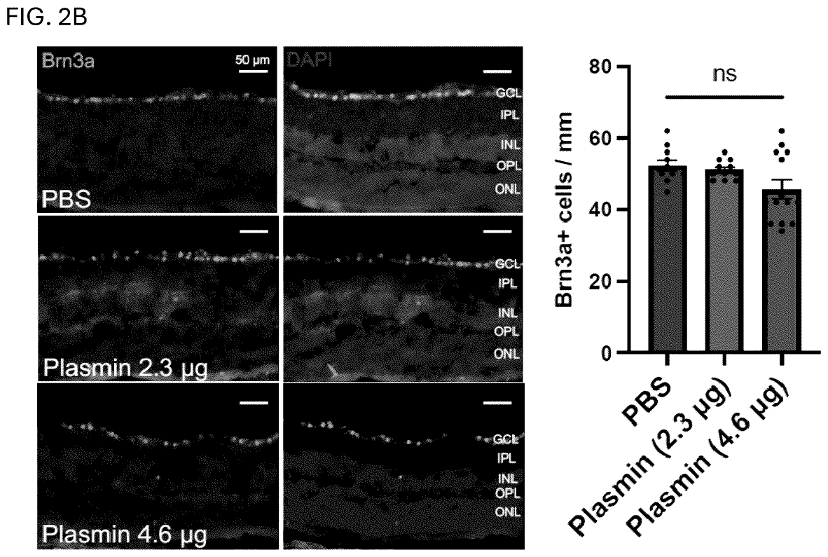

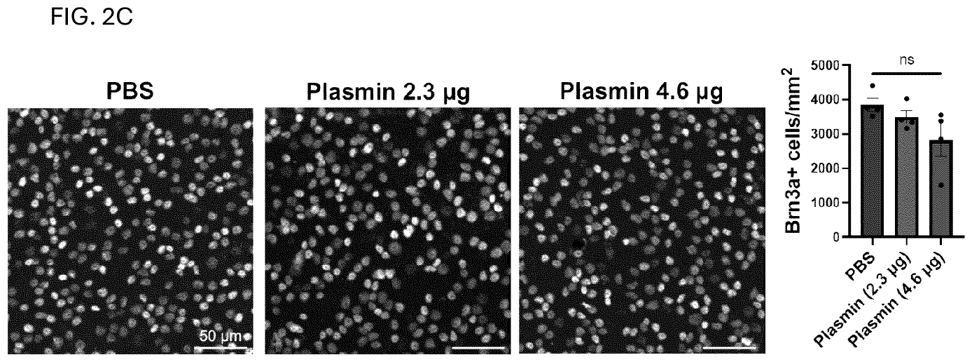

After investigating the effects on immune response, it was determined whether plasmin could effectively disrupt the ILM. For this reason, laminin staining was performed to visualize the ILM. The data showed that both 2.3 μg and 4.6 μg of plasmin treatment disrupt the structural integrity of the ILM, as proven by the interruption of laminin expression, which is a major component of the ILM. The immunofluorescence of laminin at the ILM was quantified using the quantitative method by Zhang et al (Zhang et al., Stem Cell Reports. Jan. 12, 2021; 16(1):149-167). Linear percentage coverage of the retinal surface in cryosections was measured showing that plasmin after 2 weeks reduces the coverage of ILM by 40% (FIG. 2A). Both doses of plasmin demonstrated the same percent of disruption.

Consequently, and since the anatomical location of the RGCs is right beneath the ILM, it is important to overcome the ILM barrier without damaging them. To evaluate that, the RGC marker Brn3a was used to immunostain retinal cryosections and wholemounts after 2 weeks of plasmin treatment. These results demonstrated that neither the 2.3 μg nor the 4.6 μg group showed a significant reduction in RGC numbers (FIGS. 2B and 2C). Overall, these results show that intravitreal plasmin treatment is capable of disrupting the ILM, while maintaining the RGC population numbers.

### Example 4

### Retinal Function Cam be Reduced, while Ganglion Cell Complex Thickness Demonstrated No Changes

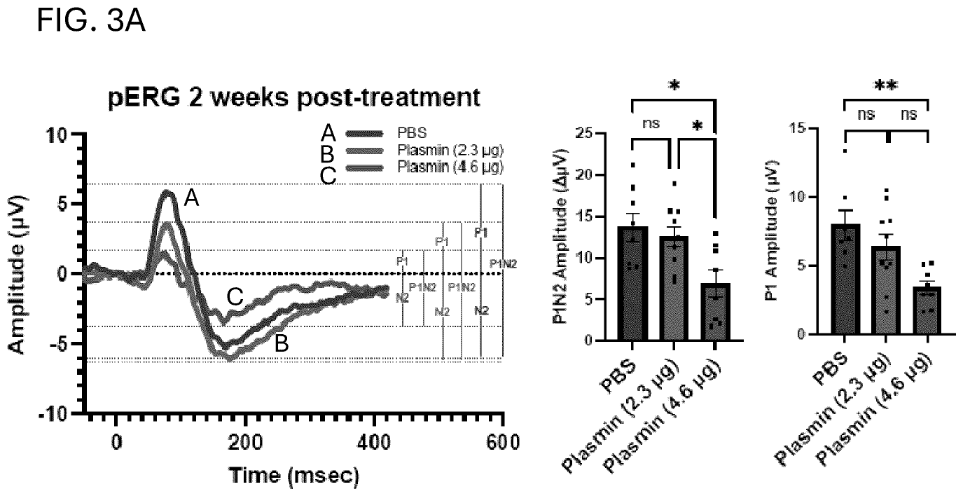

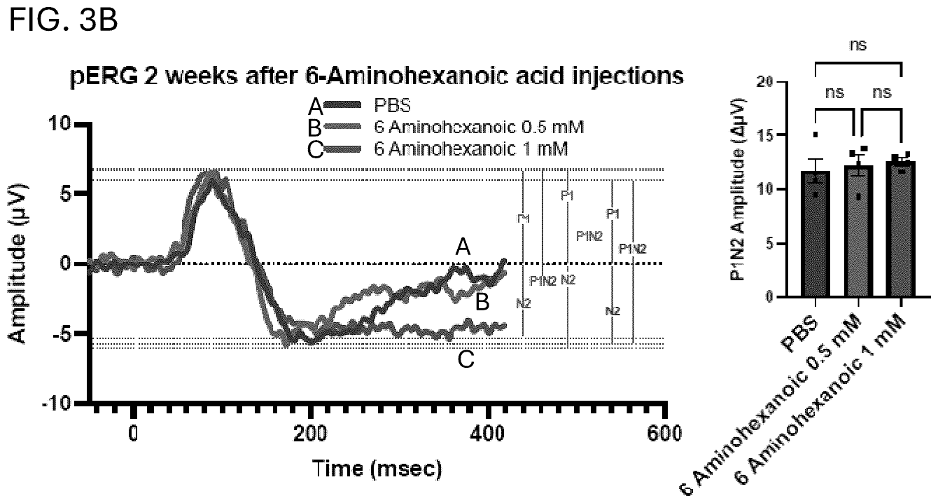

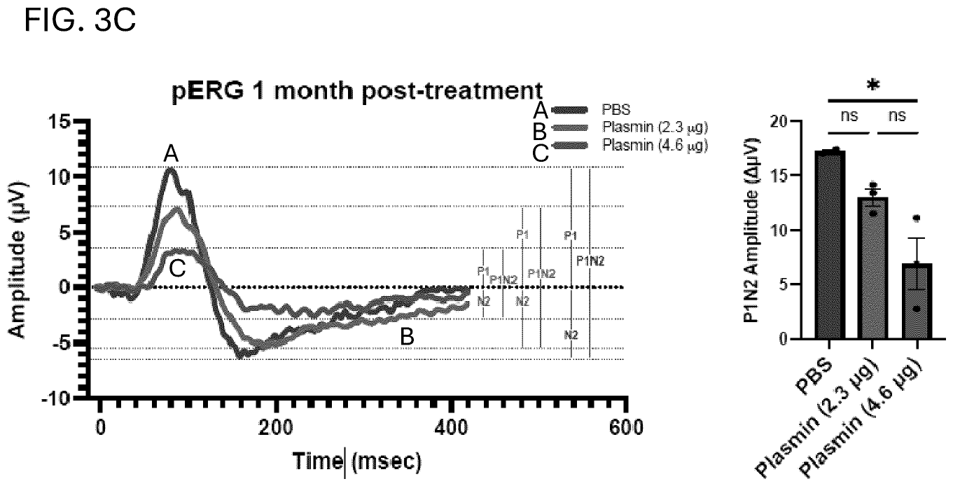

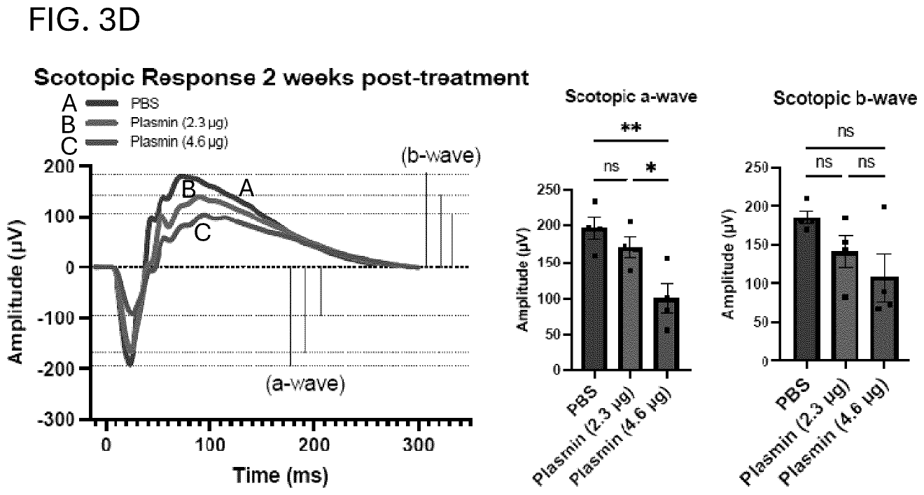

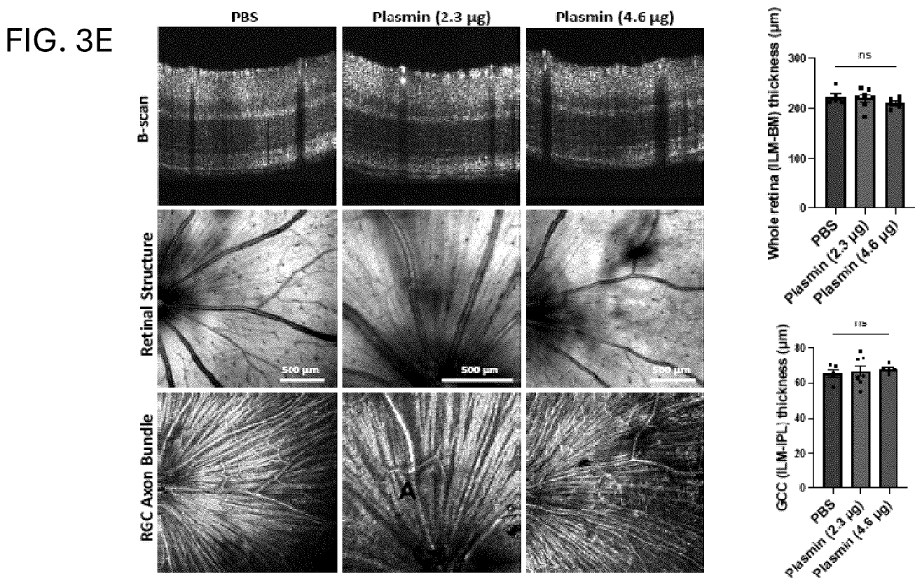

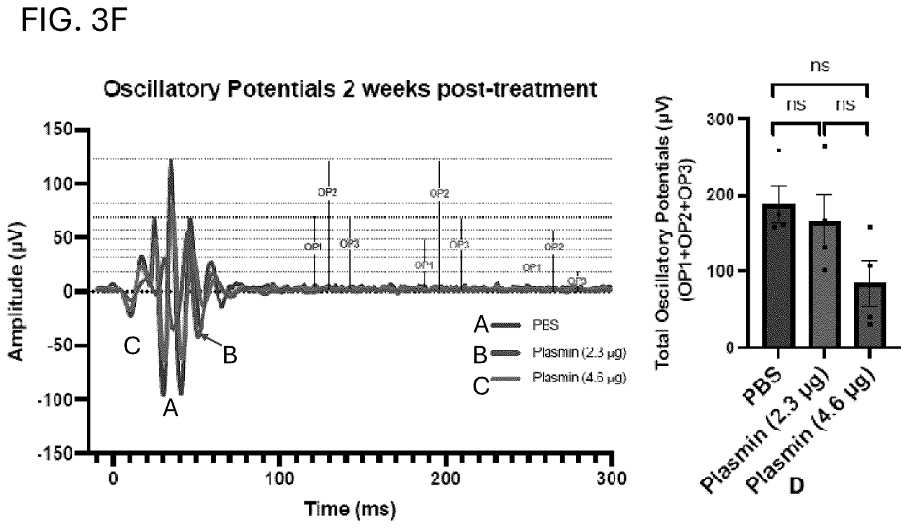

It was determined whether RGCs maintained their functional integrity following plasmin treatment. To assess this, pERG recordings were conducted on PBS- and plasmin-treated mice 2 weeks after treatment (FIG. 3A). pERG is an effective noninvasive method for evaluating RGC function, as its generation relies on the physiological response of existing RGCs to a reversing black-and-white checkerboard pattern, with the response diminishing in cases of RGC degeneration (Fung et al., Cochrane Database Syst Rev. Mar. 15, 2024; 3(3):CD015514). There was no significant difference in total amplitude between the plasmin 2.3 μg and PBS groups, indicating that RGCs remained physiologically viable and that 2.3 μg plasmin treatment did not cause measurable functional damage. In contrast, the total amplitude in the plasmin 4.6 μg group was significantly decreased, demonstrating impairment of function. Additionally, the analysis of the P1 wave showed significant decrease in the 4.6 μg group compared to the PBS. To ensure that these findings were not attributable to the 6-aminohexanoic acid present as a stabilizing component in the commercial plasmin preparation, the mice were assigned to three groups: PBS, 0.5 mM 6-aminohexanoic acid (equivalent to the concentration present in the 2.3 μg plasmin dose), and 1 mM 6-aminohexanoic acid (equivalent to the concentration in the 4.6 μg plasmin dose). Analysis of pERG recordings revealed no differences in overall response amplitudes among the groups, indicating that the observed effects are not driven by 6-aminohexanoic acid itself (FIG. 3B). Moreover, to assess the long-term effects of plasmin, pERG was performed 1 month after injection, revealing that the 4.6 μg group continued to show a significant reduction in RGC function (FIG. 3C). Next, the photoreceptors' functional integrity was tested using ffERG (FIG. 3D), which records the summed electrical response of the retina to uniform light flashes and is widely used to evaluate overall retinal function (Robson et al., Doc Ophthalmol. June 2022; 144(3):165-177). Scotopic a- and b-waves were analyzed from the ffERG and the a-wave amplitude was significantly reduced in the 4.6 μg group compared with both the PBS and 2.3 μg groups, indicating decreased photoreceptor activity, predominantly from rods. In contrast, scotopic b-wave amplitudes showed no statistically significant differences among the three groups, suggesting preserved bipolar cell function. Additionally, optical coherence tomography (OCT) scans, an imaging modality for in vivo, noninvasive, and longitudinal monitoring of the retina for both rodents and humans (Pi et al., Invest Ophthalmol Vis Sci. Apr. 3, 2023; 64(4):17), were performed two weeks after the injections (FIG. 3E). While no apparent differences in retinal structure were observed among the groups, the texture of the RGC axon bundle in the plasmin 4.6 μg-treated eyes appeared less distinct compared to the PBS and plasmin 2.3 μg groups, suggesting that 4.6 μg of plasmin may induce a certain degree of damage to the RGCs. The impact of plasmin treatment on retinal thickness was also assessed. Two weeks after administering PBS or plasmin, the ganglion cell complex (GCC) thickness was measured from the ILM to the inner plexiform layer (IPL), as well as the total retinal thickness from the ILM to Bruch's membrane (BM). No significant differences were observed between the groups. Oscillatory potentials exhibited a trend toward reduction between the PBS and 4.6 μg groups (p=0.08) but did not reach statistical significance, implying maintained inner retinal activity, particularly from bipolar and amacrine cells (FIG. 3F). Overall, the data demonstrate that the 2.3 μg of plasmin is better tolerated than the 4.6 μg dose, as it neither reduces the number of RGCs nor impairs their function.

### Example 5

### The Survival of Transplanted Cells is Increased One Week after Transplantation in the Plasmin-Treated Eyes

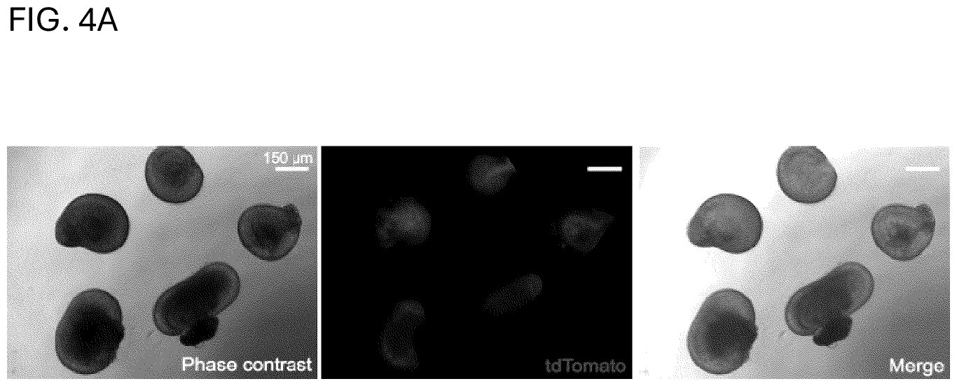

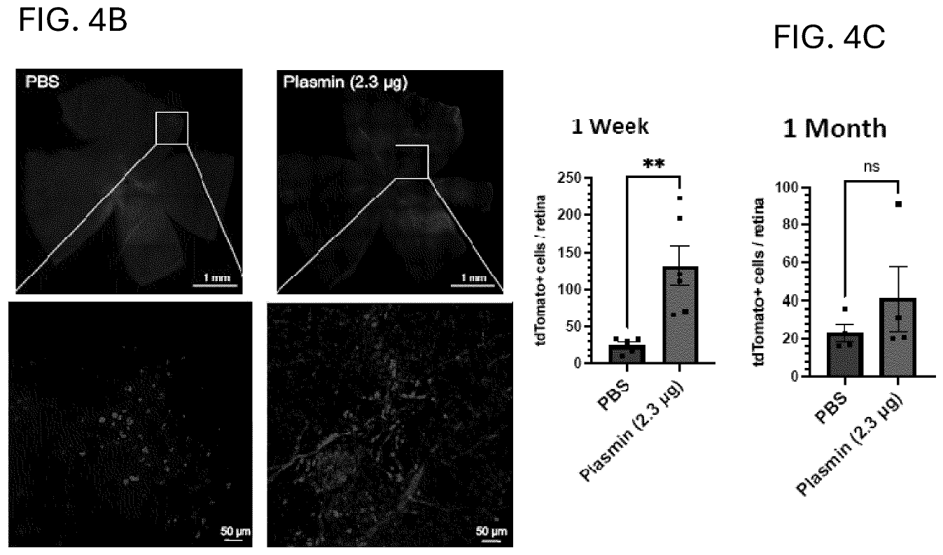

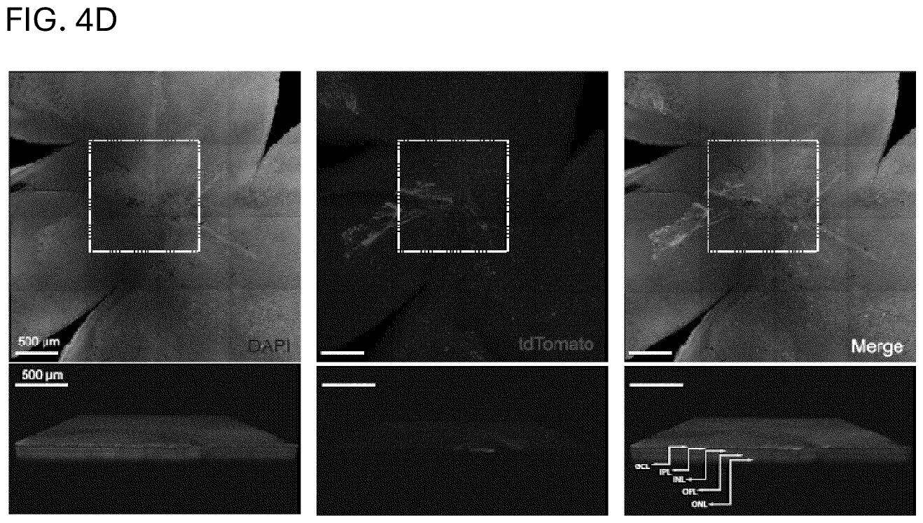

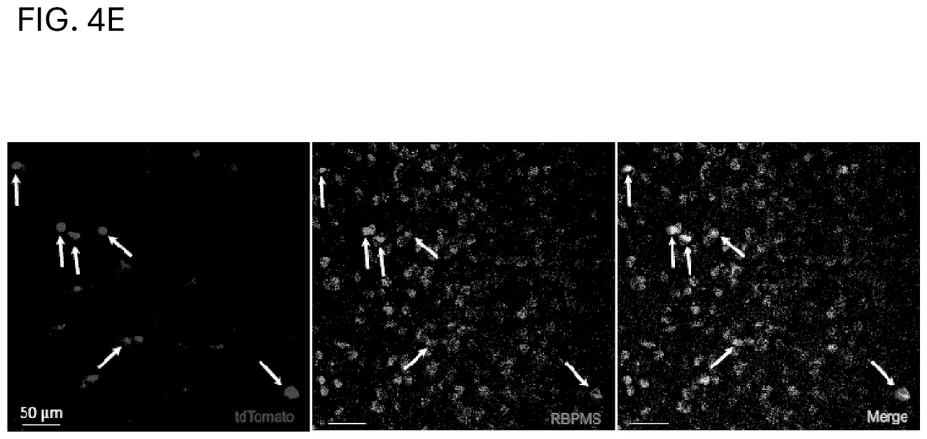

It was investigated whether ILM disruption following plasmin-treatment would have impact on donor cell survival. To obtain human RGCs, human BRN3B-H9-tdTomato-Thy1.2 human embryonic stem cells were differentiated into human retinal organoids (hROs). In this study, tdTomato fluorescence was observed in the center of the day 60 retinal organoids indicating RGC formation (FIG. 4A). To transplant donor cells, day 60 retinal organoids were dissociated and approximately 20,000 cells were intravitreally injected into mouse eyes. Dissociated human retinal organoids were transplanted in PBS and plasmin 2.3 μg-treated retinas. One week after donor cell transplantation more tdTomato+ human RGCs were observed on the plasmin-compared to the PBS-injected retinas suggesting increased donor cells survival (FIG. 4B). However, one month after cell transplantation no significant difference in cell survivability was observed between the two groups (FIG. 4C). By using 3D Z-projection image analysis, tdTomato+ donor neurites were observed in the ganglion cell layer one week after transplantation (FIG. 4D). Later, the immunostaining data of one month transplantation experiments showed that donor tdTomato+ cells were stained with RBPMS on wholemount retinas, as the same layer of endogenous RBPMS+ cells, confirming their RGC identity (FIG. 4E). These data suggest that the transplanted cells potentially migrated in the host retina's ganglion cell layer.

In this study, plasmin was utilized to induce ILM digestion in mice in vivo. The disclosed studies revealed that the application of 2.3 μg of plasmin effectively disrupts the ILM without inducing significant ocular inflammation, reducing RGC numbers or impairing their function. OCT scans conducted two weeks after plasmin administration showed no changes in GCC or total retinal thickness. This was consistent in both the 2.3 μg and the 4.6 μg plasmin groups. Additionally, the survival of donor cells is increased one week after transplantation in plasmin treated-retinas and the potential integration of donor cells within the host retina was demonstrated (without significant observation of extensive neurite growth).

In rodent models, pluripotent stem cell-derived RGCs have been successfully transplanted into mouse retinas with donor cells surviving for up to 12 months. These cells developed axonal processes that co-aligned with host axons and extended into the optic nerve head, demonstrating structural integration (Oswald et al., Ther Methods Clin Dev. Jun. 11, 2021; 21:180-198). Additionally, studies using primary RGC transplants in adult rat retinas have demonstrated light-evoked responses in donor cells, providing direct functional evidence of integration (Venugopalan et al., Nat Commun. Feb. 4, 2016; 7:10472). These findings indicate that functional incorporation is feasible when donor cells survive and integrate appropriately. As disclosed herein, enhanced donor cell survival following plasmin treatment can increase the likelihood of such light-responsive outcomes. Immunosuppression may be beneficial to reduce loss of transplanted cells. The disclosed studies provide evidence that ocriplasmin, plasmin, or plasminogen can be administered to an eye of the subject to increase the integration of RGCs in the retina.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

We claim:

1. A method of increasing integration of transplanted retinal ganglion cells in a retina of a subject, comprising:
administering an effective amount of ocriplasmin, plasmin, or plasminogen to an eye of the subject; and
transplanting an effective amount of retinal ganglion cells into the eye of the subject, thereby increasing the integration of retinal ganglion cells in the retina in the eye of the subject.

2. A method of treating glaucoma or optic neuropathy in an eye of a subject thereof, comprising:
administering an effective amount of ocriplasmin, plasmin, or plasminogen to the eye of the subject; and
transplanting an effective amount of retinal ganglion cells into the eye of the subject, thereby treating the glaucoma or optic neuropathy in the eye of a subject.

3. The method of claim 1, wherein the subject is human, and the retinal ganglion cells are human cells.

4. The method of claim 1, wherein the subject has optic neuropathy.

5. The method of claim 1, wherein the subject has glaucoma.

6. The method of claim 5, wherein the glaucoma is normo-tensive glaucoma.

7. The method of claim 5, further comprising administering an effective amount of an agent that lowers intraocular pressure to the subject.

8. The method of claim 7, wherein the agent that lowers intraocular pressure is a) a prostaglandin analog, b) a beta-adrenergic blocker, c) an alpha-adrenergic agonist, or d) a cholinergic agonist.

9. The method of claim 8, wherein the agent that lowers intraocular pressure is a) latanoprost, b) timolol, c) brimonidine, or d) pilocarpine.

10. The method of claim 1, wherein the retinal ganglion cells are produced from stem cells in vitro prior to transplanting the retinal ganglion cells.

11. The method of claim 10, wherein the stem cells are induced pluripotent stem cells.

12. The method of claim 10, wherein the stem cells are autologous.

13. The method of claim 1, comprising administering to the subject the effective amount of plasmin.

14. The method of claim 13, comprising administering to the eye of the subject a) about 3 internal international units (IU) to about 7 IU of plasmin; b) about 4 to about 6 IU of plasmin; or c) about 5 IU of plasmin.

15. The method of claim 1, comprising administering the effective amount of ocriplasmin, plasmin, or plasminogen to the eye of the subject about 1 to about 3 weeks prior to transplanting the effective amount of retinal ganglion cells to the eye of the subject.

16. The method of claim 15, comprising administering the effective amount of ocriplasmin, plasmin, or plasminogen to the eye of the subject about 2 weeks prior to transplanting the effective amount of retinal ganglion cells to the eye of the subject.

17. The method of claim 15, wherein the retinal ganglion cells are in an organoid.

18. The method of claim 1, wherein the effective amount of ocriplasmin, plasmin, or plasminogen is administered via injection into the vitreous of the eye of the subject.

19. The method of claim 1, wherein the method increases the survival of the retinal ganglion cells in the subject.

20. The method of claim 1, further comprising administering to the subject an effective amount of an immunosuppressive agent.

21. The method of claim 1, wherein
a) administering an effective amount of ocriplasmin, plasmin, or plasminogen to an eye of the subject comprises intravitral administration; and/or
b) transplanting the effective amount of retinal ganglion cells to the eye of the subject comprises intravitreal administration.
